SEQUENCE LISTING (244 sequences)

SEQ ID NO: 1 - DNA, 20 bp, Artificial Sequence
  Synthetic
ccgcccctcctagccagctg

SEQ ID NO: 2 - DNA, 20 bp, Artificial Sequence
  Synthetic
gaggcagtgatagatgatgg

SEQ ID NO: 3 - DNA, 20 bp, Artificial Sequence
  Synthetic
cgggtaccgagagtaggtgg

SEQ ID NO: 4 - DNA, 20 bp, Artificial Sequence
  Synthetic
atcaggaccagctcgggcag

SEQ ID NO: 5 - DNA, 20 bp, Artificial Sequence
  Synthetic
gtgcctcttggctctccagt

SEQ ID NO: 6 - DNA, 20 bp, Artificial Sequence
  Synthetic
ggcgcctggatgccggcccg

SEQ ID NO: 7 - DNA, 20 bp, Artificial Sequence
  Synthetic
gaggtaacagagtggccccg

SEQ ID NO: 8 - DNA, 20 bp, Artificial Sequence
  Synthetic
gcccagacatccgcagcccg

SEQ ID NO: 9 - DNA, 20 bp, Artificial Sequence
  Synthetic
ccccatcttactgcaaggcc

SEQ ID NO: 10 - DNA, 20 bp, Artificial Sequence
  Synthetic
ccccatcttactgcaaggcc

SEQ ID NO: 11 - DNA, 20 bp, Artificial Sequence
  Synthetic
ccccatcttactgcaaggcc

SEQ ID NO: 12 - DNA, 20 bp, Artificial Sequence
  Synthetic
ctgggtgtgaggtcccgcag

SEQ ID NO: 13 - DNA, 20 bp, Artificial Sequence
  Synthetic
atcaggaccagctcgggcag

SEQ ID NO: 14 - DNA, 20 bp, Artificial Sequence
  Synthetic
atcaggaccagctcgggcag

SEQ ID NO: 15 - DNA, 20 bp, Artificial Sequence
  Synthetic
ccccatcttactgcaaggcc

SEQ ID NO: 16 - DNA, 20 bp, Artificial Sequence
  Synthetic
ccccatcttactgcaaggcc

SEQ ID NO: 17 - DNA, 20 bp, Artificial Sequence
  Synthetic
tatttccctacaatatcgca

SEQ ID NO: 18 - DNA, 20 bp, Artificial Sequence
  Synthetic
atgagcccacaactggggga

SEQ ID NO: 19 - DNA, 20 bp, Artificial Sequence
  Synthetic
atatgtgactgtccttgaag

SEQ ID NO: 20 - DNA, 20 bp, Artificial Sequence
  Synthetic
gagaactttaatttaccaag

SEQ ID NO: 21 - DNA, 20 bp, Artificial Sequence
  Synthetic
ggagatcaatgggcaggagc

SEQ ID NO: 22 - DNA, 20 bp, Artificial Sequence
  Synthetic
gaagatatttccatgtctgc

SEQ ID NO: 23 - DNA, 20 bp, Artificial Sequence
  Synthetic
cctggattctagatcatagg

SEQ ID NO: 24 - DNA, 20 bp, Artificial Sequence
  Synthetic
gagatgaccacttagaccac

SEQ ID NO: 25 - DNA, 20 bp, Artificial Sequence
  Synthetic
ccgcccccaccggcagaccc

SEQ ID NO: 26 - DNA, 20 bp, Artificial Sequence
  Synthetic
tacaggtgtcataaaggacg

SEQ ID NO: 27 - DNA, 20 bp, Artificial Sequence
  Synthetic
tgtgggataagatcgcggag

SEQ ID NO: 28 - DNA, 20 bp, Artificial Sequence
  Synthetic
gcccccaccggcagaccccg

SEQ ID NO: 29 - DNA, 20 bp, Artificial Sequence
  Synthetic
cagcgttacagtgaaacctg

SEQ ID NO: 30 - DNA, 20 bp, Artificial Sequence
  Synthetic
gcgctaactccagagccaga

SEQ ID NO: 31 - DNA, 20 bp, Artificial Sequence
  Synthetic
ctgggaggttgaacattctg

SEQ ID NO: 32 - DNA, 20 bp, Artificial Sequence
  Synthetic
gtccatgccaacacgaggcc

SEQ ID NO: 33 - DNA, 20 bp, Artificial Sequence
  Synthetic
accatctcttaactcagcag

SEQ ID NO: 34 - DNA, 20 bp, Artificial Sequence
  Synthetic
cctcaggttggcaactactg

SEQ ID NO: 35 - DNA, 20 bp, Artificial Sequence
  Synthetic
ctgagtattctacaacccca

SEQ ID NO: 36 - DNA, 20 bp, Artificial Sequence
  Synthetic
gtccatgccaacacgaggcc

SEQ ID NO: 37 - DNA, 20 bp, Artificial Sequence
  Synthetic
gtccatgccaacacgaggcc

SEQ ID NO: 38 - DNA, 20 bp, Artificial Sequence
  Synthetic
gcttccagagacacctacag

SEQ ID NO: 39 - DNA, 20 bp, Artificial Sequence
  Synthetic
gcttccagagacacctacag

SEQ ID NO: 40 - DNA, 20 bp, Artificial Sequence
  Synthetic
gcttccagagacacctacag

SEQ ID NO: 41 - DNA, 20 bp, Artificial Sequence
  Synthetic
accatctcttaactcagcag

SEQ ID NO: 42 - DNA, 20 bp, Artificial Sequence
  Synthetic
cccggggccagagcagttag

SEQ ID NO: 43 - DNA, 20 bp, Artificial Sequence
  Synthetic
cccggggccagagcagttag

SEQ ID NO: 44 - DNA, 20 bp, Artificial Sequence
  Synthetic
ctgagtattctacaacccca

SEQ ID NO: 45 - DNA, 20 bp, Artificial Sequence
  Synthetic
gtcgttgaggccagcgggcg

SEQ ID NO: 46 - DNA, 20 bp, Artificial Sequence
  Synthetic
gcgacagcagccaaggatcg

SEQ ID NO: 47 - DNA, 20 bp, Artificial Sequence
  Synthetic
gagccagctggttcttgtgt

SEQ ID NO: 48 - DNA, 20 bp, Artificial Sequence
  Synthetic
gctggttcttgtgtaggccg

SEQ ID NO: 49 - DNA, 20 bp, Artificial Sequence
  Synthetic
tcacagaggaggcactgtgg

SEQ ID NO: 50 - DNA, 20 bp, Artificial Sequence
  Synthetic
cctggggtctgagtgcaaac

SEQ ID NO: 51 - DNA, 20 bp, Artificial Sequence
  Synthetic
cagtcgttgaggccagcggg

SEQ ID NO: 52 - DNA, 20 bp, Artificial Sequence
  Synthetic
ccatgtcttgggacctgctg

SEQ ID NO: 53 - DNA, 20 bp, Artificial Sequence
  Synthetic
gttgaggccagcgggcgggg

SEQ ID NO: 54 - DNA, 20 bp, Artificial Sequence
  Synthetic
gacatggtcaatcccgggca

SEQ ID NO: 55 - DNA, 20 bp, Artificial Sequence
  Synthetic
agccctggatcccctgggag

SEQ ID NO: 56 - DNA, 20 bp, Artificial Sequence
  Synthetic
cagtcgttgaggccagcggg

SEQ ID NO: 57 - DNA, 20 bp, Artificial Sequence
  Synthetic
atccctgcctgcacgtgggg

SEQ ID NO: 58 - DNA, 20 bp, Artificial Sequence
  Synthetic
gttcaggggatacttcctgc

SEQ ID NO: 59 - DNA, 20 bp, Artificial Sequence
  Synthetic
tcatgtgcgtggcctgctgg

SEQ ID NO: 60 - DNA, 20 bp, Artificial Sequence
  Synthetic
tcatgtgcgtggcctgctgg

SEQ ID NO: 61 - DNA, 20 bp, Artificial Sequence
  Synthetic
ctcctgggtatctttacggc

SEQ ID NO: 62 - DNA, 20 bp, Artificial Sequence
  Synthetic
gtgacagtgtcaccagtaag

SEQ ID NO: 63 - DNA, 20 bp, Artificial Sequence
  Synthetic
ctcctgggtatctttacggc

SEQ ID NO: 64 - DNA, 20 bp, Artificial Sequence
  Synthetic
gtgacagtgtcaccagtaag

SEQ ID NO: 65 - DNA, 20 bp, Artificial Sequence
  Synthetic
agtggtaggagggcacccgt

SEQ ID NO: 66 - DNA, 20 bp, Artificial Sequence
  Synthetic
taactgtgactcatgagaga

SEQ ID NO: 67 - DNA, 20 bp, Artificial Sequence
  Synthetic
tactgtccctactgtaaagg

SEQ ID NO: 68 - DNA, 20 bp, Artificial Sequence
  Synthetic
actggcatcggcgctagcgt

SEQ ID NO: 69 - DNA, 20 bp, Artificial Sequence
  Synthetic
tgccaagccatgctagactg

SEQ ID NO: 70 - DNA, 20 bp, Artificial Sequence
  Synthetic
gtatgtcaatgtttaaactg

SEQ ID NO: 71 - DNA, 20 bp, Artificial Sequence
  Synthetic
ggcgctagcgttggtagcag

SEQ ID NO: 72 - DNA, 20 bp, Artificial Sequence
  Synthetic
ctgaatatctatggacctgg

SEQ ID NO: 73 - DNA, 20 bp, Artificial Sequence
  Synthetic
tgccaagccatgctagactg

SEQ ID NO: 74 - DNA, 20 bp, Artificial Sequence
  Synthetic
ctgaatatctatggacctgg

SEQ ID NO: 75 - DNA, 20 bp, Artificial Sequence
  Synthetic
tgccaagccatgctagactg

SEQ ID NO: 76 - DNA, 20 bp, Artificial Sequence
  Synthetic
ctgtcaacccacataattgg

SEQ ID NO: 77 - DNA, 20 bp, Artificial Sequence
  Synthetic
agaactccatccttccatgg

SEQ ID NO: 78 - DNA, 20 bp, Artificial Sequence
  Synthetic
tgccaagccatgctagactg

SEQ ID NO: 79 - DNA, 20 bp, Artificial Sequence
  Synthetic
aaattatgttaagactgggg

SEQ ID NO: 80 - DNA, 20 bp, Artificial Sequence
  Synthetic
agaactccatccttccatgg

SEQ ID NO: 81 - DNA, 20 bp, Artificial Sequence
  Synthetic
tggtgaagctgcatgggcca

SEQ ID NO: 82 - DNA, 20 bp, Artificial Sequence
  Synthetic
gacaaaacaagtctcaatgg

SEQ ID NO: 83 - DNA, 20 bp, Artificial Sequence
  Synthetic
agggaggtgcaccaaaacct

SEQ ID NO: 84 - DNA, 20 bp, Artificial Sequence
  Synthetic
cgagacaggggaaccccgag

SEQ ID NO: 85 - DNA, 20 bp, Artificial Sequence
  Synthetic
ggacattgtaggtgctccca

SEQ ID NO: 86 - DNA, 20 bp, Artificial Sequence
  Synthetic
gacctctgccctctgtgcca

SEQ ID NO: 87 - DNA, 20 bp, Artificial Sequence
  Synthetic
ataggagcgccctggcccag

SEQ ID NO: 88 - DNA, 20 bp, Artificial Sequence
  Synthetic
gtctgggcaagcaagatccc

SEQ ID NO: 89 - DNA, 20 bp, Artificial Sequence
  Synthetic
gggccgcatgcggaccctag

SEQ ID NO: 90 - DNA, 20 bp, Artificial Sequence
  Synthetic
cgagacaggggaaccccgag

SEQ ID NO: 91 - DNA, 20 bp, Artificial Sequence
  Synthetic
ggatccgcacagcggccctg

SEQ ID NO: 92 - DNA, 20 bp, Artificial Sequence
  Synthetic
ataggagcgccctggcccag

SEQ ID NO: 93 - DNA, 20 bp, Artificial Sequence
  Synthetic
tagggtccgcatgcggcccg

SEQ ID NO: 94 - DNA, 20 bp, Artificial Sequence
  Synthetic
gggccgcatgcggaccctag

SEQ ID NO: 95 - DNA, 20 bp, Artificial Sequence
  Synthetic
gtgcagaggtcacgagacag

SEQ ID NO: 96 - DNA, 20 bp, Artificial Sequence
  Synthetic
ggatccgcacagcggccctg

SEQ ID NO: 97 - DNA, 20 bp, Artificial Sequence
  Synthetic
gtggatagaggagcgggtag

SEQ ID NO: 98 - DNA, 20 bp, Artificial Sequence
  Synthetic
gaatcaatgaaaagatcagg

SEQ ID NO: 99 - DNA, 20 bp, Artificial Sequence
  Synthetic
actggctctccaatttgctg

SEQ ID NO: 100 - DNA, 20 bp, Artificial Sequence
  Synthetic
actggctctccaatttgctg

SEQ ID NO: 101 - DNA, 20 bp, Artificial Sequence
  Synthetic
gaaagagcaaacctgaaggg

SEQ ID NO: 102 - DNA, 20 bp, Artificial Sequence
  Synthetic
gcagcagtgaatatgtgcac

SEQ ID NO: 103 - DNA, 20 bp, Artificial Sequence
  Synthetic
tcgcatggtgaatcatgtca

SEQ ID NO: 104 - DNA, 20 bp, Artificial Sequence
  Synthetic
gagagtggctcttgcacctg

SEQ ID NO: 105 - DNA, 20 bp, Artificial Sequence
  Synthetic
ttggaagacgatggcatcag

SEQ ID NO: 106 - DNA, 20 bp, Artificial Sequence
  Synthetic
tgtgctggaggtgtcacagt

SEQ ID NO: 107 - DNA, 20 bp, Artificial Sequence
  Synthetic
cagtttattccacaccgctg

SEQ ID NO: 108 - DNA, 20 bp, Artificial Sequence
  Synthetic
cgctgcacagccagtcccag

SEQ ID NO: 109 - DNA, 20 bp, Artificial Sequence
  Synthetic
ttgcaggctgcctcaagccc

SEQ ID NO: 110 - DNA, 20 bp, Artificial Sequence
  Synthetic
gaacaggctttccttgatgg

SEQ ID NO: 111 - DNA, 20 bp, Artificial Sequence
  Synthetic
ctggtggctgcacgctccaa

SEQ ID NO: 112 - DNA, 20 bp, Artificial Sequence
  Synthetic
gtaccagcctggatctcttg

SEQ ID NO: 113 - DNA, 20 bp, Artificial Sequence
  Synthetic
gacaggctgagctagtgagc

SEQ ID NO: 114 - DNA, 20 bp, Artificial Sequence
  Synthetic
aactccagccatctgtcccg

SEQ ID NO: 115 - DNA, 20 bp, Artificial Sequence
  Synthetic
ggtggagttctgaaacagct

SEQ ID NO: 116 - DNA, 20 bp, Artificial Sequence
  Synthetic
ggtgaagagccccgaaggaa

SEQ ID NO: 117 - DNA, 20 bp, Artificial Sequence
  Synthetic
gacaggctgagctagtgagc

SEQ ID NO: 118 - DNA, 20 bp, Artificial Sequence
  Synthetic
gcttccttgatgtcaaccct

SEQ ID NO: 119 - DNA, 20 bp, Artificial Sequence
  Synthetic
gtaccagcctggatctcttg

SEQ ID NO: 120 - DNA, 20 bp, Artificial Sequence
  Synthetic
ggtggagttctgaaacagct

SEQ ID NO: 121 - DNA, 20 bp, Artificial Sequence
  Synthetic
tgtgctggaggtgtcacagt

SEQ ID NO: 122 - DNA, 20 bp, Artificial Sequence
  Synthetic
gaacaggctttccttgatgg

SEQ ID NO: 123 - DNA, 20 bp, Artificial Sequence
  Synthetic
ggcttcggcttaatgaactg

SEQ ID NO: 124 - DNA, 20 bp, Artificial Sequence
  Synthetic
ggcttcggcttaatgaactg

SEQ ID NO: 125 - DNA, 20 bp, Artificial Sequence
  Synthetic
ggtggagttctgaaacagct

SEQ ID NO: 126 - DNA, 20 bp, Artificial Sequence
  Synthetic
cgctgcacagccagtcccag

SEQ ID NO: 127 - DNA, 20 bp, Artificial Sequence
  Synthetic
atccaccccgaatttaggca

SEQ ID NO: 128 - DNA, 20 bp, Artificial Sequence
  Synthetic
gggtacacgcaatggaggca

SEQ ID NO: 129 - DNA, 20 bp, Artificial Sequence
  Synthetic
gaacctgaatgacttaggga

SEQ ID NO: 130 - DNA, 20 bp, Artificial Sequence
  Synthetic
aatgaatgcggcttgagtag

SEQ ID NO: 131 - DNA, 20 bp, Artificial Sequence
  Synthetic
tacagaaacgatgtagttgg

SEQ ID NO: 132 - DNA, 20 bp, Artificial Sequence
  Synthetic
tcctaatcagcagaaaaggg

SEQ ID NO: 133 - DNA, 20 bp, Artificial Sequence
  Synthetic
caacttggataggaataaga

SEQ ID NO: 134 - DNA, 20 bp, Artificial Sequence
  Synthetic
accccaccatgtttaattag

SEQ ID NO: 135 - DNA, 20 bp, Artificial Sequence
  Synthetic
gagctagggcatactttagg

SEQ ID NO: 136 - DNA, 20 bp, Artificial Sequence
  Synthetic
ggccagtcttgctttctggg

SEQ ID NO: 137 - DNA, 20 bp, Artificial Sequence
  Synthetic
ttggataggaataagacgga

SEQ ID NO: 138 - DNA, 20 bp, Artificial Sequence
  Synthetic
ataaagggtacacgcaatgg

SEQ ID NO: 139 - DNA, 20 bp, Artificial Sequence
  Synthetic
actcaaagttgatattaaag

SEQ ID NO: 140 - DNA, 20 bp, Artificial Sequence
  Synthetic
caacttggataggaataaga

SEQ ID NO: 141 - DNA, 20 bp, Artificial Sequence
  Synthetic
gggtacacgcaatggaggca

SEQ ID NO: 142 - DNA, 20 bp, Artificial Sequence
  Synthetic
caacttggataggaataaga

SEQ ID NO: 143 - DNA, 20 bp, Artificial Sequence
  Synthetic
ttgagttagcaagtaactat

SEQ ID NO: 144 - DNA, 20 bp, Artificial Sequence
  Synthetic
ggccagtcttgctttctggg

SEQ ID NO: 145 - DNA, 20 bp, Artificial Sequence
  Synthetic
ttcagatcagattagtacag

SEQ ID NO: 146 - DNA, 20 bp, Artificial Sequence
  Synthetic
tcctaatcagcagaaaaggg

SEQ ID NO: 147 - DNA, 20 bp, Artificial Sequence
  Synthetic
tcctaatcagcagaaaaggg

SEQ ID NO: 148 - DNA, 20 bp, Artificial Sequence
  Synthetic
ttgagttagcaagtaactat

SEQ ID NO: 149 - DNA, 20 bp, Artificial Sequence
  Synthetic
tgctcgtggaataacacaaa

SEQ ID NO: 150 - DNA, 20 bp, Artificial Sequence
  Synthetic
cttaaacaaggttgggaggg

SEQ ID NO: 151 - DNA, 20 bp, Artificial Sequence
  Synthetic
aatggaaccagcttgaacct

SEQ ID NO: 152 - DNA, 20 bp, Artificial Sequence
  Synthetic
gtggaataacacaaagggcc

SEQ ID NO: 153 - DNA, 20 bp, Artificial Sequence
  Synthetic
cgcagtgtgagaggagccta

SEQ ID NO: 154 - DNA, 20 bp, Artificial Sequence
  Synthetic
caatacggcatttcttcaag

SEQ ID NO: 155 - DNA, 20 bp, Artificial Sequence
  Synthetic
aatggaaccagcttgaacct

SEQ ID NO: 156 - DNA, 20 bp, Artificial Sequence
  Synthetic
caatggggtaacaactcctg

SEQ ID NO: 157 - DNA, 20 bp, Artificial Sequence
  Synthetic
caatacggcatttcttcaag

SEQ ID NO: 158 - DNA, 20 bp, Artificial Sequence
  Synthetic
aatggaaccagcttgaacct

SEQ ID NO: 159 - DNA, 20 bp, Artificial Sequence
  Synthetic
gcatctcacccccttaaccc

SEQ ID NO: 160 - DNA, 20 bp, Artificial Sequence
  Synthetic
gccgccggggtaggttcgag

SEQ ID NO: 161 - DNA, 20 bp, Artificial Sequence
  Synthetic
cctctcagcactcgctaccg

SEQ ID NO: 162 - DNA, 20 bp, Artificial Sequence
  Synthetic
actacttcatccctctgggc

SEQ ID NO: 163 - DNA, 20 bp, Artificial Sequence
  Synthetic
gctgatgattcatggtgcgc

SEQ ID NO: 164 - DNA, 20 bp, Artificial Sequence
  Synthetic
acaacacgtgaccccggaga

SEQ ID NO: 165 - DNA, 20 bp, Artificial Sequence
  Synthetic
tgcaatgacagcccagagcg

SEQ ID NO: 166 - DNA, 20 bp, Artificial Sequence
  Synthetic
aggagggcaacgaggccggc

SEQ ID NO: 167 - DNA, 20 bp, Artificial Sequence
  Synthetic
ggctacattgctccccagag

SEQ ID NO: 168 - DNA, 20 bp, Artificial Sequence
  Synthetic
ggctacattgctccccagag

SEQ ID NO: 169 - DNA, 20 bp, Artificial Sequence
  Synthetic
ggctacattgctccccagag

SEQ ID NO: 170 - DNA, 20 bp, Artificial Sequence
  Synthetic
ggctacattgctccccagag

SEQ ID NO: 171 - DNA, 20 bp, Artificial Sequence
  Synthetic
ggctacattgctccccagag

SEQ ID NO: 172 - DNA, 20 bp, Artificial Sequence
  Synthetic
ggctacattgctccccagag

SEQ ID NO: 173 - DNA, 20 bp, Artificial Sequence
  Synthetic
ggctacattgctccccagag

SEQ ID NO: 174 - DNA, 20 bp, Artificial Sequence
  Synthetic
ggctacattgctccccagag

SEQ ID NO: 175 - DNA, 20 bp, Artificial Sequence
  Synthetic
cctcgtggccgctcaggccc

SEQ ID NO: 176 - DNA, 20 bp, Artificial Sequence
  Synthetic
ggtcctgccttcaggctccg

SEQ ID NO: 177 - DNA, 20 bp, Artificial Sequence
  Synthetic
ctccggaggcgcccggaaag

SEQ ID NO: 178 - DNA, 20 bp, Artificial Sequence
  Synthetic
cggaggcgcccggaaagagg

SEQ ID NO: 179 - DNA, 20 bp, Artificial Sequence
  Synthetic
acagcccttggtggacatgg

SEQ ID NO: 180 - DNA, 20 bp, Artificial Sequence
  Synthetic
tcttatgtagcattgtggaa

SEQ ID NO: 181 - DNA, 20 bp, Artificial Sequence
  Synthetic
cgggggggtgaactatctgt

SEQ ID NO: 182 - DNA, 20 bp, Artificial Sequence
  Synthetic
cgggggggtgaactatctgt

SEQ ID NO: 183 - DNA, 20 bp, Artificial Sequence
  Synthetic
gatggatatgtacaatcaag

SEQ ID NO: 184 - DNA, 20 bp, Artificial Sequence
  Synthetic
gcgattcagaaataaagctt

SEQ ID NO: 185 - DNA, 20 bp, Artificial Sequence
  Synthetic
ttgtgtaggtgatcaagcca

SEQ ID NO: 186 - DNA, 20 bp, Artificial Sequence
  Synthetic
ggtcttataagagtgggtga

SEQ ID NO: 187 - DNA, 20 bp, Artificial Sequence
  Synthetic
ggctaaatcctccaattggg

SEQ ID NO: 188 - DNA, 20 bp, Artificial Sequence
  Synthetic
ataccagagttgtcatgagg

SEQ ID NO: 189 - DNA, 20 bp, Artificial Sequence
  Synthetic
ggatgagctccctccccaag

SEQ ID NO: 190 - DNA, 20 bp, Artificial Sequence
  Synthetic
gaatgaggactagagggggc

SEQ ID NO: 191 - DNA, 20 bp, Artificial Sequence
  Synthetic
tgtgtgacagctcaggccag

SEQ ID NO: 192 - DNA, 20 bp, Artificial Sequence
  Synthetic
agtccctcctgcccacaagg

SEQ ID NO: 193 - DNA, 20 bp, Artificial Sequence
  Synthetic
acatggatgctaccaaccac

SEQ ID NO: 194 - DNA, 20 bp, Artificial Sequence
  Synthetic
gtcaggccaagcatgcaggc

SEQ ID NO: 195 - DNA, 20 bp, Artificial Sequence
  Synthetic
atcagcatggatgcctgcaa

SEQ ID NO: 196 - DNA, 20 bp, Artificial Sequence
  Synthetic
aggggctggtgctctgccgc

SEQ ID NO: 197 - DNA, 20 bp, Artificial Sequence
  Synthetic
atcagcatggatgcctgcaa

SEQ ID NO: 198 - DNA, 20 bp, Artificial Sequence
  Synthetic
tgagtcgatcctgcgcacag

SEQ ID NO: 199 - DNA, 20 bp, Artificial Sequence
  Synthetic
acaacctgtccccacacgtg

SEQ ID NO: 200 - DNA, 20 bp, Artificial Sequence
  Synthetic
catggaaataccaggtgctg

SEQ ID NO: 201 - DNA, 20 bp, Artificial Sequence
  Synthetic
catggaaataccaggtgctg

SEQ ID NO: 202 - DNA, 20 bp, Artificial Sequence
  Synthetic
acaacctgtccccacacgtg

SEQ ID NO: 203 - DNA, 20 bp, Artificial Sequence
  Synthetic
tgttggtgctgagtgcggat

SEQ ID NO: 204 - DNA, 20 bp, Artificial Sequence
  Synthetic
ctcagttcgtaaacactcag

SEQ ID NO: 205 - DNA, 20 bp, Artificial Sequence
  Synthetic
tgttcgtgccgcacaaatag

SEQ ID NO: 206 - DNA, 20 bp, Artificial Sequence
  Synthetic
tctctaatgtgtaaagtaag

SEQ ID NO: 207 - DNA, 20 bp, Artificial Sequence
  Synthetic
tacctgattcatgttacagc

SEQ ID NO: 208 - DNA, 20 bp, Artificial Sequence
  Synthetic
gggaagccgccagtatatgg

SEQ ID NO: 209 - DNA, 20 bp, Artificial Sequence
  Synthetic
gtccgggaaggtctttgtca

SEQ ID NO: 210 - DNA, 20 bp, Artificial Sequence
  Synthetic
ggtggacgcaagcttggtcc

SEQ ID NO: 211 - DNA, 20 bp, Artificial Sequence
  Synthetic
gggcactgttaagtggaaaa

SEQ ID NO: 212 - DNA, 20 bp, Artificial Sequence
  Synthetic
agcttggtcccagaggggag

SEQ ID NO: 213 - DNA, 20 bp, Artificial Sequence
  Synthetic
gtccgggaaggtctttgtca

SEQ ID NO: 214 - DNA, 20 bp, Artificial Sequence
  Synthetic
ggactcaagcttggtcccag

SEQ ID NO: 215 - DNA, 20 bp, Artificial Sequence
  Synthetic
agcttggtcccagaggggag

SEQ ID NO: 216 - DNA, 20 bp, Artificial Sequence
  Synthetic
ggtggacacatgcttagctg

SEQ ID NO: 217 - DNA, 20 bp, Artificial Sequence
  Synthetic
ggactcaagcttggtcccag

SEQ ID NO: 218 - DNA, 20 bp, Artificial Sequence
  Synthetic
ggtggacgcaagcttggtcc

SEQ ID NO: 219 - DNA, 20 bp, Artificial Sequence
  Synthetic
agtgcatgaagccggaggga

SEQ ID NO: 220 - DNA, 20 bp, Artificial Sequence
  Synthetic
ggtggacacatgcttagctg

SEQ ID NO: 221 - DNA, 20 bp, Artificial Sequence
  Synthetic
ggactcaagcttggtcccag

SEQ ID NO: 222 - DNA, 20 bp, Artificial Sequence
  Synthetic
actcaggggtaacatgggga

SEQ ID NO: 223 - DNA, 20 bp, Artificial Sequence
  Synthetic
ctagctaggaaactgcggag

SEQ ID NO: 224 - DNA, 20 bp, Artificial Sequence
  Synthetic
tatcttatctcaataggaaa

SEQ ID NO: 225 - DNA, 20 bp, Artificial Sequence
  Synthetic
ggactctcgctcctcgcggg

SEQ ID NO: 226 - DNA, 20 bp, Artificial Sequence
  Synthetic
gtggagaccccatctcatga

SEQ ID NO: 227 - DNA, 20 bp, Artificial Sequence
  Synthetic
agtagggcgcaagatgtcgg

SEQ ID NO: 228 - DNA, 20 bp, Artificial Sequence
  Synthetic
tcaagtccggagcagaggcg

SEQ ID NO: 229 - DNA, 20 bp, Artificial Sequence
  Synthetic
agaattcatgctgtgagaac

SEQ ID NO: 230 - DNA, 20 bp, Artificial Sequence
  Synthetic
gaggcacgccacacaaccct

SEQ ID NO: 231 - DNA, 20 bp, Artificial Sequence
  Synthetic
tccatttctcaatacgccag

SEQ ID NO: 232 - DNA, 20 bp, Artificial Sequence
  Synthetic
ttatctccttaaaaaccgag

SEQ ID NO: 233 - DNA, 20 bp, Artificial Sequence
  Synthetic
cagccgatggaaaattcaga

SEQ ID NO: 234 - DNA, 20 bp, Artificial Sequence
  Synthetic
gtagttcactgtggatgaag

SEQ ID NO: 235 - DNA, 20 bp, Artificial Sequence
  Synthetic
cagccgatggaaaattcaga

SEQ ID NO: 236 - DNA, 20 bp, Artificial Sequence
  Synthetic
tctcttcatcatactgaggg

SEQ ID NO: 237 - DNA, 20 bp, Artificial Sequence
  Synthetic
agtagggcgcaagatgtcgg

SEQ ID NO: 238 - DNA, 20 bp, Artificial Sequence
  Synthetic
cacgtgttctgctcaaaaga

SEQ ID NO: 239 - DNA, 20 bp, Artificial Sequence
  Synthetic
tccatagttggaaaatgtgg

SEQ ID NO: 240 - DNA, 20 bp, Artificial Sequence
  Synthetic
ctacgaacactggcagccga

SEQ ID NO: 241 - DNA, 20 bp, Artificial Sequence
  Synthetic
tccatagttggaaaatgtgg

SEQ ID NO: 242 - DNA, 20 bp, Artificial Sequence
  Synthetic
gtagttcactgtggatgaag

SEQ ID NO: 243 - DNA, 20 bp, Artificial Sequence
  Synthetic
cagccgatggaaaattcaga

SEQ ID NO: 244 - DNA, 20 bp, Artificial Sequence
  Synthetic
gggccgagtgctggctgccc

(19) United States
(12) Patent Application Publication
(10) Pub. No.: US 2024/0093190 A1
(43) Pub. Date: Mar. 21, 2024

(54) ENGINEERED NUCLEIC ACIDS TARGETING LONG NONCODING RNA INVOLVED IN PATHOGENIC INFECTION

(71) Applicant: President and Fellows of Harvard College, Cambridge, MA (US)

(72) Inventors: Longlong Si, Cambridge, MA (US); Haiqing Bai, Cambridge, MA (US); Rachelle Pranti-Baun, Cambridge, MA (US); Donald E. Ingber, Cambridge, MA (US)

(73) Assignee: President and Fellows of Harvard College, Cambridge, MA (US)

(21) Appl. No.: 18/272,933

(22) PCT Filed: Jan. 18, 2022
(86) PCT No.: PCT/US2022/012764

§ 371 Date: Jul. 18, 2023

Related U.S. Application Data
(60) Provisional application No. 63/138,836, filed on Jan. 19, 2021.

(51) Int. Cl. C12N 15/113; C12N 9/22

(52) U.S. Cl. CPC C12N 15/113; C12N 9/22; C12N 2310/113; C12N 2310/20; C12N 2320/12

(57) ABSTRACT

The present disclosure provides compositions and methods for inhibiting viral pathogenesis by targeting long noncoding ribonucleic acids.

### RELATED APPLICATION

This application claims the benefit under 35 U.S.C. § 119(e) of U.S. provisional application Ser. No. 63/138,836, filed Jan. 19, 2021, which is incorporated by reference herein in its entirety.

### GOVERNMENT FUNDING

This invention was made with Government support under HL141797 awarded by National Institutes of Health and W911NF-12-2-0036 awarded by Department of Defense/DARPA. The government has certain rights in the invention.

### BACKGROUND

Respiratory viruses are the most frequent causative agents of disease in humans, impacting morbidity and mortality worldwide. Common respiratory agents from several virus families are well adapted to efficient person-to-person transmission and circulate globally. Community-based studies have confirmed that these viruses are the predominant etiological agents of acute respiratory infections. The respiratory viruses that most commonly circulate as endemic or epidemic agents are influenza virus, respiratory syncytial virus, parainfluenza viruses, metapneumovirus, rhinovirus, coronaviruses, adenoviruses, and bocaviruses. Vaccines and effective antiviral drugs are not yet available for most of these viruses.

### SUMMARY

The present disclosure provides, in some aspects, engineered nucleic acids encoding or comprising an inhibitory oligonucleotide that targets (e.g., binds to) long non-coding RNAs (lncRNAs) involved in the pathogenesis of respiratory viruses, such as influenza viruses and betacoronaviruses. Also provided herein, in some aspects, are pharmaceutical compositions comprising the engineered nucleic acids and methods of using the engineered nucleic acids, for example, to inhibit respiratory virus pathogenesis, including infection and propagation.

Identifying the cellular factors involved in respiratory virus infection and understanding their roles is critical for exploring the mechanism of viral pathogenesis and developing new antiviral therapies. Most investigations to date have focused on the host proteins translated from coding regions of genome, however, the majority (˜98%) of the genome is transcribed as noncoding RNAs, which include a rich subset of long noncoding RNAs (lncRNAs). Recent advances in the high-throughput sequencing techniques have provided the tools needed to identify lncRNAs that are involved in infections and immunological processes; however, the role of cellular lncRNAs in respiratory virus (e.g., influenza virus) pathogenesis remains relatively unexplored.

The data provided herein demonstrate that certain lncRNAs, for example, DiGeorge Syndrome Critical Region Gene 5 (DGCR5) lncRNA, are involved in respiratory virus infection in human lung epithelial cells. Knockdown of the lncRNAs, in some instances, activates the interferon pathway, which results in up-regulation of type I and II interferons that are known to inhibit viral infection.

Thus, some aspects of the present disclosure provide a method of inhibiting respiratory virus pathogenesis in a subject, comprising administering to a subject an engineered nucleic acid encoding or comprising an inhibitory oligonucleotide that targets a long non-coding RNA (lncRNA) of any one of Tables 1-2, or any one of those listed in Table 3 of Zhu S et al. Nat Biotechnol. 2016 December; 34(12):1279-1286 (incorporated herein by reference), wherein the subject is infected with or at risk of infection with a respiratory virus.

In some embodiments, the administering upregulates a type I interferon pathway in the subject. In some embodiments, the administering inhibits pathogenesis in the subject, optionally by reducing pathogen titer.

Some aspects of the present disclosure provide an engineered nucleic acid encoding or comprising an inhibitory oligonucleotide that targets a long non-coding RNA (lncRNA) of any one of Tables 1-2, or any one of those listed in Table 3 of Zhu S et al. Nat Biotechnol. 2016 December; 34(12):1279-1286, optionally for use in a method of inhibiting respiratory virus pathogenesis.

In some embodiments, the lncRNA is involved in pathogenesis of a virus. In some embodiments, the lncRNA is involved in viral infection and/or propagation.

In some embodiments, the lncRNA is utilized by a pathogen to enhance propagation of the pathogen.

In some embodiments, the virus is a respiratory virus. For example, the respiratory virus may be selected from the group consisting of an influenza virus (e.g., A/WSN/33 (H1N1), influenza A/Hong Kong/8/68 (H3N2), or influenza A/Avian Influenza (H5N1)), a coronavirus (e.g., betacoronavirus, e.g., SARS-CoV-2), a rhinovirus, an enterovirus, a parainfluenza virus, a metapneumovirus, a respiratory syncytial virus, an adenovirus, and a bocavirus.

In some embodiments, the lncRNA is selected from the group consisting of: DGCR5, AC015987.1, LINC01146, LRRC37A11P, LINC00176, PCAT7, CECR7, MIR503HG, RFPL1S, CYP4A22-AS1, CTC-498J12.1, RP11-360F5.1, LINC00885, LINC00086, GS1-124K5.11, CTD-2127H9.1, RP11-475N22.4, AC108488.4, and TMEM44-AS1 (See Table 2).

In some embodiments, the lncRNA is selected from the group consisting of: DGCR5, AC015987.1, LINC01146, AR, LRRC37A11P, RPL36, AAVS1, LINC00176, FOXA1, PCAT7, CECR7, RSL24D1, MIR503HG, RFPL1S, CYP4A22-AS1, RP5-107303.2, TPT1-AS1, RP11-548L20.1, LINC01060, RP1-122P22.2, AC093375.1, LINC00844, CCDC183-AS1, RP1-734K21.5, AC104135.2, CTC-527H23.3, H19, ANKRD18CP, RP11-70F11.8, RP11-167H9.6, RP6-65G23.3, RAP2C-AS1, RP11-128M1.1, RP11-76N22.2, RPL21, LINC00639, LINC00657, CTD-2541M15.1, LINC01087, MAPKAPK5-AS1, RP11-195M16.1, AC005329.7, CSAG4, RP11-760H22.2, RP1-179N16.6, RP11-333113.1, RP11-435O5.2, AC084809.2, CTD-2566J3.1, AC009478.1, CTB-181F24.1, RP11-308D16.4, RP11-314C16.1, AC020571.3, RP11-725D20.1, RP11-367G18.1, LINC01132, HOXB13, RP1-462P6.1, RP5-1142A6.9, FTX, LINC00471, RP11-498P14.5, RP11-318M2.2, CTD-2587M2.1, RP11-304F15.7, DLGAP1-AS2, RP11-299G20.2, RP11-789C1.1, RPL14, RP11-151A6.4, RP11-627G23.1, CTD-2016O11.1, ENTPD1-AS1, AE000661.37, RP11-134G8.8, SNHG5, EZH2, RPL37A, CTD-3051D23.4, LINC00925, RP11-732M18.3, JRK, RP11-802E16.3, LINC00984, EGOT, RPL39, RP11-473M20.14, TGGENE, RP11-15I11.2, RP11-677M14.3, RP11-170M17.1, RP11-65J3.1, RP1-97O12.7, SNAI3-AS1, AC095067.1, LINC01133, RP11-540A21.2, RP1-261D10.2, RP11-268G12.1, RP11-90K6.1, RP11-373N22.3, RP11-394O4.3, LINC00205, RP11-399D6.2, RP1-400K9.4, RP11-96D1.7, KB-1460A1.1, LINC00277, and RP11-269F19.2.

In some embodiments, the lncRNA is DiGeorge Syndrome Critical Region Gene 5 (DGCR5).

In some embodiments, the engineered nucleic acid comprises DNA. In other embodiments, the engineered nucleic acid comprises RNA. In other embodiments, the engineered nucleic acid comprises DNA and RNA.

In some embodiments, the engineered nucleic acid is single stranded. In other embodiments, the engineered nucleic acid is double stranded. In yet other embodiments, the engineered nucleic acid is partially double-stranded.

In some embodiments, the inhibitory oligonucleotide inhibits expression and/or function of the lncRNA (e.g., by at least 10%, 20%, 30%, 40%, or 50% relative to a control).

A control, as provided herein, may be lncRNA expression in the absence of an inhibitory oligonucleotide.

In some embodiments, the inhibitory oligonucleotide binds to the lncRNA (e.g., targeting DGCR5). In other embodiments, the inhibitory oligonucleotide binds to the lncRNA or binds to DNA encoding the lncRNA (e.g., targeting DGCR5).

In some embodiments, the inhibitory oligonucleotide is a clustered regularly interspaced short palindromic repeats (CRISPR) guide RNA (gRNA), for example, a Cas9 gRNA or a Cas13 gRNA (e.g., targeting DGCR5).

In some embodiments, the gRNA comprises a sequence having at least 90% (at least 91%, at least 92%, at least 93%, at least 94%, at least 95%, at least 96%, at least 97%, at least 98%, at least 99%, or 100%) identity to a gRNA sequence of Table 1. In some embodiments, the gRNA comprises a sequence having at least 90% (at least 91%, at least 92%, at least 93%, at least 94%, at least 95%, at least 96%, at least 97%, at least 98%, at least 99%, or 100%) identity to a gRNA sequence of any one of SEQ ID NOs: 1-16 (e.g., targeting DGCR5).

In some embodiments, the gRNA comprises a sequence having at least 90% (at least 91%, at least 92%, at least 93%, at least 94%, at least 95%, at least 96%, at least 97%, at least 98%, at least 99%, or 100%) identity to a gRNA sequence of any one of SEQ ID NOs: 1-244.

In some embodiments, the inhibitory oligonucleotide is an antisense oligonucleotide (ASO) (e.g., targeting DGCR5).

In some embodiments, the inhibitory oligonucleotide is an RNA interference molecule (e.g., targeting DGCR5). For example, the RNA interference molecule may be selected from the group consisting of a small interfering RNA (siRNA), a microRNA (miRNA), and a short hairpin RNA (shRNA).

Other aspects of the present disclosure provide a vector comprising the engineered nucleic acid of any one of the preceding claims. In some embodiments, the vector is selected from the group consisting of a plasmid, a phagemid, a cosmid, and a viral vector.

Yet aspects of the present disclosure provide a nanoparticle comprising the engineered nucleic acid of any one of the preceding claims. In some embodiments, the nanoparticle is a lipid nanoparticle.

Still aspects of the present disclosure provide a pharmaceutical composition comprising the engineered nucleic acid, vector, or nanoparticle of any one of the preceding paragraphs and a pharmaceutically-acceptable excipient.

Some aspects of the present disclosure provide a method comprising administering to a subject the engineered nucleic acid, vector, nanoparticle, or pharmaceutical composition of any one of the preceding paragraphs. In some embodiments, the subject is infected with or at risk of infection with a pathogen. The subject may be, for example, a human subject. In some embodiments, the administration is intravenous, intramuscular, intraperitoneal, subcutaneous, or intranasal.

### BRIEF DESCRIPTION OF THE DRAWINGS

FIG. 1 shows a schematic of CRISPR-Cas9 deletion technology-based screening for influenza-associated lncRNAs.

FIGS. 2A-2C show the discovery of lncRNA DGCR5 whose knock down decreased influenza virus infection. FIG. 2A shows A549 cells that were transfected with siRNAs (IDT lnc) to knockdown DGCR5. 24 h later, cells were infected with GFP-labeled influenza A/PR8/34 (H1N1) virus (MOI=0.01). GFP signals were recorded 48 h post-infection. Scramble siRNAs were used as a control. FIG. 2B shows A549 cells that were transfected with siRNAs (IDT lnc) to knockdown DGCR5. 24 h later, cells were infected with influenza A/WSN/33 (H1N1) virus (MOI=0.01). Supernatants were collected for viral titer detection by plaque formation assay. Scramble siRNAs were used as control. FIG. 2C shows human airway chips were transfected with siRNAs (IDT lnc) to knockdown DGCR5. 24 h later, cells were infected with influenza A/WSN/33 (H1N1) virus (MOI=0.01). Samples were collected for viral NP gene detection by RT-qPCR. Scramble siRNAs were used as control. ***, P<0.001.

FIG. 3 shows the effect of influenza infection on the level of lncRNA DGCR5 in A549 cells. A549 cells were infected with influenza A/WSN/33 (H1N1) virus. 48 h later, cells were collected for lncRNA DGCR5 detection by RT-qPCR. *, P<0.05; **, P<0.01.

FIGS. 4A-4D show DGCR5 is a negative regulator of type I interferon (IFN-1) pathways. FIG. 4A shows a volcano plot of differentially expressed genes (DEGs) from RNA-seq after knockdown of DGCR5. FIG. 4B shows GO Enrichment analysis for DEGs. FIG. 4C shows a volcano plot of differentiated expressed proteins from TMT mass spectrometry after knockdown of DGCR5. FIG. 4D shows GO Enrichment analysis of differentiated expressed proteins.

FIG. 5 shows the knockout of IRF3 abolished the effect of DGCR5 on IFN-1 pathway. Wild-type HAP1 cells, IRF7-knockout HAP1 cells, or IRF3 knockout HAP1 cells were transfected with siRNAs (IDT lnc) to knock down DGCR5. 48 h later, cells were collected for detection of genes of IFN-1 pathway, including STAT1, IL4L1, TRAIL, IFFI6 and IFN-β1, by RT-qPCR. Scramble siRNAs were used as control.

FIG. 6 shows a schematic of the role of lncRNA DGCR5.

### DETAILED DESCRIPTION

The present disclosure provides compositions and methods for inhibiting pathogenesis of a respiratory pathogen (e.g., virus), such as an influenza virus or a betacoronavirus. As shown herein, a gene-editing-based genome-wide platform technology was used to identify respiratory virus-associated lncRNAs that serve as targets for developing therapeutics for respiratory virus infection, for example. The studies herein identified DGCR5 as a new lncRNA associated with influenza virus pathogenesis—knocking down DGCR5 upregulated type I interferon-IRF3 pathway and inhibited influenza virus infection. The IFN-I pathway is involved in many diseases, including infection of pathogens (e.g., viruses, bacteria, fungi, and parasites), cancers, and autoimmune diseases; thus modulating DGCR5 lncRNA and other lncRNAs involved in the IFN-I pathway, for example, provides a new therapeutic strategy for intervention of these diseases.

Host lncRNA Targets

The present disclosure identifies host lncRNAs that mediate pathogenesis of a virus (e.g., respiratory virus, such as influenza virus or coronavirus). Pathogenesis refers to the processes by which a pathogen (e.g., virus, bacteria, fungus, etc.) causes disease in a host. The term “pathogenesis” herein encompasses pathogen infection, propagation (replication/reproduction) and survival in a host.

Accordingly, in some embodiments, provided herein are engineered nucleic acids encoding or comprising inhibitory oligonucleotides that target a lncRNA (e.g., at least 2, 3, 4, 5, 6, 7, 8, 9, or 10 lncRNAs) in a host (e.g., human subject). In some embodiments, an engineered nucleic acid encoding or comprising an inhibitory oligonucleotide prevents pathogen (e.g., viral) infection and/or reduces pathogen (e.g., viral) titer in a host, relative to a control (e.g., pathogen viral titer in the absence of the inhibitory oligonucleotide, also referred to as baseline viral titer).

In some embodiments, the lncRNA target is selected from those listed in Table 2 or a variant thereof. For example, the host lncRNA target may be selected from the group consisting of: DGCR5, AC015987.1, LINC01146, LRRC37A11P, PCAT7, CECR7, MIR503HG, RFPL1S, CYP4A22-AS1, CTC-498J12.1, RP11-360F5.1, LINC00885, LINC00086, GS1-124K5.11, CTD-2127H9.1, AC108488.4, and TMEM44-AS1 (see, e.g., Tables 1 and 2). In some embodiments, the host incRNA target is DGCR5.

DGCR5 is a lncRNA located on chromosome 22q11 and is associated with DiGeorge syndrome. As shown here, knocking down (reducing/elimination expression and/or function of) DGCR5 inhibits influenza replication. Without wishing to be bound by theory, knockdown of DGCR5 activates the interferon pathway, which results in up-regulation of type I and II interferons that are known to inhibit viral infection. Accordingly, in some aspects, the disclosure provides a method of inhibiting a viral pathogenesis (e.g., influenza infection) by targeting DGCR5.

In some embodiments, the disclosure provides a method of inhibiting a viral infection (e.g., influenza infection) in a subject in need thereof, comprising administering to the subject an agent that inhibits DGCR5 (e.g., an inhibitory oligonucleotide, a small molecule inhibitor, etc.).

In some embodiments, the disclosure provides a method of reducing viral titer in a subject in need thereof, comprising administering to the subject an agent that inhibits DGCR5 (e.g., an inhibitory oligonucleotide, a small molecule inhibitor, etc.).

In some embodiments, the incRNA target is selected from those listed in Table 2 or a variant thereof. For example, the host lncRNA target may be selected from the group consisting of: DGCR5, AC015987.1, LINC01146, AR, LRRC37A11P, RPL36, AAVS1, LINC00176, FOXA1, PCAT7, CECR7, RSL24D1, MIR503HG, RFPL1S, CYP4A22-AS1, RP5-107303.2, TPT1-AS1, RP11-548L20.1, LINC01060, RP1-122P22.2, AC093375.1, LINC00844, CCDC183-AS1, RP11-734K21.5, AC104135.2, CTC-527H23.3, H19, ANKRD18CP, RP11-70F11.8, RP11-167H9.6, RP6-65G23.3, RAP2C-AS1, RP11-128M1.1, RP11-76N22.2, RPL21, LINC00639, LINC00657, CTD-2541M15.1, LINC01087, MAPKAPK5-AS1, RP11-195M16.1, AC005329.7, CSAG4, RP11-760H22.2, RP1-179N16.6, RP11-333113.1, RP11-435O5.2, AC084809.2, CTD-2566J3.1, AC009478.1, CTB-181F24.1, RP11-308D16.4, RP11-314C16.1, AC020571.3, RP11-725D20.1, RP11-367G18.1, LINC01132, HOXB13, RP11-462P6.1, RP5-1142A6.9, FTX, LINC00471, RP11-498P14.5, RP11-318M2.2, CTD-2587M2.1, RP11-304F15.7, DLGAP1-AS2, RP11-299G20.2, RP11-789C1.1, RPL14, RP11-151A6.4, RP11-627G23.1, CTD-2016O11.1, ENTPD1-AS1, AE000661.37, RP11-134G8.8, SNHG5, EZH2, RPL37A, CTD-3051D23.4, LINC00925, RP11-732M18.3, JRK, RP11-802E16.3, LINC00984, EGOT, RPL39, RP11-473M20.14, TGGENE, RP11-15I11.2, RP11-677M14.3, RP11-170M17.1, RP11-65J3.1, RP11-97O12.7, SNAI3-AS1, AC095067.1, LINC01133, RP11-540A21.2, RP1-261D10.2, RP11-268G12.1, RP11-90K6.1, RP11-373N22.3, RP11-394O4.3, LINC00205, RP11-399D6.2, RP11-400K9.4, RP11-96D1.7, KB-1460A1.1, LINC00277, and RP11-269F19.2.

### Inhibitory Oligonucleotides

Aspects of the disclosure provide engineered nucleic acids comprising or encoding an inhibitory oligonucleotide that targets (e.g., binds to) a lncRNA involved in pathogenesis of a virus. It should be understood that the terms “nucleic acid” and “oligonucleotide” may be used interchangeably herein. An engineered nucleic acid is a nucleic acid (e.g., at least two nucleotides covalently linked together, and in some instances, containing phosphodiester bonds, referred to as a phosphodiester backbone) that does not occur in nature. Engineered nucleic acids include recombinant nucleic acids and synthetic nucleic acids. A recombinant nucleic acid is a molecule that is constructed by joining nucleic acids (e.g., isolated nucleic acids, synthetic nucleic acids or a combination thereof) from two different organisms (e.g., human and mouse). A synthetic nucleic acid is a molecule that is amplified or chemically, or by other means, synthesized. A synthetic nucleic acid includes those that are chemically modified, or otherwise modified, but can base pair with (bind to) naturally occurring nucleic acid molecules. Recombinant and synthetic nucleic acids also include those molecules that result from the replication of either of the foregoing.

An engineered nucleic acid may comprise DNA (e.g., genomic DNA, cDNA or a combination of genomic DNA and cDNA), RNA or a hybrid molecule, for example, where the nucleic acid contains any combination of deoxyribonucleotides and ribonucleotides (e.g., artificial or natural), and any combination of two or more bases, including uracil, adenine, thymine, cytosine, guanine, inosine, xanthine, hypoxanthine, isocytosine and isoguanine.

Engineered nucleic acids of the present disclosure may be produced using standard molecular biology methods (see, e.g., Green and Sambrook, Molecular Cloning, A Laboratory Manual, 2012, Cold Spring Harbor Press). In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′end sequences and exposes the complementary sequence for annealing. The polymerase activity then fills in the gaps on the annealed domains. A DNA ligase then seals the nick and covalently links the DNA fragments together. The overlapping sequence of adjoining fragments is much longer than those used in Golden Gate Assembly, and therefore results in a higher percentage of correct assemblies. Other methods of producing engineered nucleic acids may be used in accordance with the present disclosure.

A promoter is a nucleotide sequence to which RNA polymerase binds to initial transcription (e.g., ATG). Promoters are typically located directly upstream from (at the 5′ end of) a transcription initiation site. In some embodiments, an engineered nucleic acid comprises a promoter operably linked to nucleotide sequence encoding an inhibitory oligonucleotide.

In some embodiments, an inhibitory oligonucleotide is chemically modified.

In some embodiments, an inhibitory oligonucleotide comprises a region of complementarity to a host lncRNA that mediates respiratory virus (e.g., influenza virus or betacoronavirus) infection. In some embodiments, an inhibitory oligonucleotide comprises a region of complementarity that shares at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% complementarity to a region of a target lncRNA.

In some embodiments, the region of complementarity (in the inhibitory oligonucleotide or in the target lncRNA) is about 4 to 50 contiguous nucleotides. In some embodiments, the region of complementarity is about 10-20 contiguous nucleotides, 15-25 contiguous nucleotides, 15-30 contiguous nucleotides, about 20-30 contiguous nucleotides, about 20-40 contiguous nucleotides, or about 30-50 contiguous nucleotides, etc. As used herein “contiguous bases” refers to two or more nucleotide bases that are covalently bound (e.g., by one or more phosphodiester bond, etc.) to each other (e.g. as part of a nucleic acid molecule). As used herein, the term “complementary” refers to the ability of polynucleotides to form base pairs with one another. Base pairs are typically formed by hydrogen bonds between nucleotide units in antiparallel polynucleotide strands or regions. Complementary polynucleotide strands or regions can base pair in the Watson-Crick manner (e.g., A to T, A to U, C to G), or in any other manner that allows for the formation of stable duplexes. It will be understood that “100% complementarity” refers to the situation in which each nucleotide unit of one polynucleotide strand or region can hydrogen bond with each nucleotide unit of a second polynucleotide strand or region. Less than 100% complementarity refers to the situation in which some, but not all, nucleotide units of two strands or two regions can hydrogen bond with each other. For example, for two 19-mers, if 17 base pairs on each strand or each region can hydrogen bond with each other, the polynucleotide strands exhibit 89.5% complementarity. In some embodiments, an inhibitory oligonucleotide may comprise one or more hairpin and/or bulge structures that are non-complementary to the target lncRNA.

In some embodiments, an inhibitory oligonucleotide of the disclosure targets a lncRNA listed in Table 1. In some embodiments, an inhibitory oligonucleotide of the disclosure targets a lncRNA listed in Table 2. In some embodiments, an inhibitory oligonucleotide of the disclosure targets a lncRNA selected from the group consisting of: DGCR5, AC015987.1, LINC01146, LRRC37A11P, PCAT7, CECR7, MIR503HG, RFPL1S, CYP4A22-AS1, CTC-498J12.1, RP11-360F5.1, LINC00885, LINC00086, GS1-124K5.11, CTD-2127H9.1, AC108488.4, and TMEM44-AS1. In some embodiments, an inhibitory oligonucleotide of the disclosure targets DGCR5.

An inhibitory oligonucleotide of the disclosure inhibits a target host lncRNA. It should be understood that the term “inhibits” encompasses complete (100%) inhibition and partial (less than 100%) inhibition, otherwise referred to as reduction. Thus, an inhibitory oligonucleotide may reduce, e.g., lncRNA expression, stability, and/or activity by at least 10%, at least 20%, at least 30%, at least 40%, at least 50%, at least 60%, at least 70%, at least 80%, at least 90%, or at least 95%, relative to a control or baseline level. In some embodiments, the control or baseline level is the expression, stability, and/or activity in the absence of the inhibitory oligonucleotide.

In some embodiments, an inhibitory oligonucleotide is about 15-120, 15-60, 15-50, 15-40 15-30, 15-25, 19-25, 20-30, or 20-24 nucleotides in length. In some embodiments, an inhibitory oligonucleotide is 15, 16, 17, 18, 19, 20, 21, 22, 23, 24, 25, 26, 27, 28, 29, 30, 31, 32, 33, 34, or 35 nucleotides in length. In some embodiments, an inhibitory oligonucleotide can also be generated by cleavage of a longer precursor nucleic acid. In some embodiments, a precursor nucleic acid is about 50-150, 60-120, 60-100, or 60-70 nucleotides in length. In some embodiments, a precursor nucleic acid is at least 50 nucleotides to about 100, 200, 300, 400, or 500 nucleotides in length. A precursor nucleic acid may be as long as 1000, 1500, 2000, 5000 nucleotides in length, or longer.

In some embodiments, an inhibitory oligonucleotide targets a lncRNA at the genomic level (i.e., DNA encoding the lncRNA). In some embodiments, the inhibitory oligonucleotide targets a host lncRNA at the RNA level.

In some embodiments, the inhibitory oligonucleotide is an antisense oligonucleotide (ASO). ASOs can target DNA or RNA.

In some embodiments, the inhibitory oligonucleotide is a CRISPR guide RNA. As is known in the art, the CRISPR pathway includes two principal components: the Cas nuclease and a guide RNA (gRNA). A gRNA is a short synthetic RNA composed of a scaffold sequence necessary for RNA-guided nuclease (e.g., Cas9, Cas12a, or Cas13) binding and a user-defined ˜20 (e.g., 20±5 or 20±10) nucleotide “spacer” or “targeting” sequence which defines the genomic target to be modified. Thus, one can change the (genomic) target of an RNA-guided nuclease (e.g., Cas9, Cas12a, or Cas13) by simply changing the targeting sequence present in the gRNA. In some embodiments, a gRNA has a length of 10 to 100 nucleotides. For example, a gRNA may have a length of 10-90, 10-80, 10-70, 10-60, 10-50, 10-40, 10-35, 10-30, 10-25, 10-20, 10-15, 15-100, 15-90, 15-80, 15-70, 15-60, 15-50, 15-40, 15-35, 15-30, 15-25, 15-20, 20-100, 20-90, 20-80, 20-70, 20-60, 20-50, 20-40, 20-35, 20-30 or 20-25 nucleotides. In some embodiments, a gRNA has a length of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides. Longer gRNAs are encompassed by the present disclosure. Methods of identifying gRNAs for use in modifying or deleting a nucleic acid sequence (e.g., of an allele) are known. For example, there are various commercial companies that offer computation programs to guide the selection of gRNA targets. See, e.g., Addgene's Validated gRNA Sequence Datatable. The general principles guiding gRNA selection include: identifying the region of the genome for targeting (the intended target site), identify protospacer sequences near the intended target site, and select protospacer sequences that minimize off-target effects. In some embodiments, a pair of gRNAs are used to delete the genomic target.

It should be understood that in any of the embodiments described herein, Cas9 nuclease may substituted with Cas12a nuclease or another CRISPR-associated nuclease (e.g., Cas13, if appropriate). In some embodiments, an engineered nucleic acid encoding a Cas nuclease is additionally provided. In some embodiments, the Cas nuclease is a Type II enzyme. In some embodiments, the Cas nuclease is a Cas9 nuclease and the guide RNA is a Cas9 guide RNA. Cas 9 nuclease and Cas12a nuclease variants are also encompassed herein. In some embodiments, the Cas nuclease is a Type III or Type VI CRISPR enzyme. Type III and Type VI CRISPR enzymes are specialized for RNA interference. In some embodiments, the Cas nuclease is Cas13 (or variant thereof) and the gRNA is a Cas13 gRNA.

In some embodiments, a gRNA comprises a nucleotide sequence that is at least 90% identical (e.g., at least 91%, at least 92%, at least 93%, at least 94%, at least 95%, at least 96%, at least 97%, at least 98%, or at least 99% identical) to a nucleotide sequence set forth set forth in Table 1. In some embodiments, a gRNA comprises a nucleotide sequence set forth in Table 1. In some embodiments, a gRNA consists of a nucleotide sequence set forth in Table 1.

In some embodiments, the gRNA comprises a sequence having at least 90% (at least 91%, at least 92%, at least 93%, at least 94%, at least 95%, at least 96%, at least 97%, at least 98%, at least 99%, or 100%) identity to a gRNA sequence of any one of SEQ ID NOs: 1-244. In some embodiments, the gRNA consists of a sequence having at least 90% (at least 91%, at least 92%, at least 93%, at least 94%, at least 95%, at least 96%, at least 97%, at least 98%, at least 99%, or 100%) identity to a gRNA sequence of any one of SEQ ID NOs: 1-16.

In some embodiments, the inhibitory oligonucleotide is an RNA interference (RNAi) molecule. Non-limiting examples of RNAi molecules include small interfering RNAs (siRNAs), microRNAs (miRNAs), and short hairpin RNAs (shRNAs).

In some embodiments, an inhibitory oligonucleotide is an siRNA. siRNAs are typically double-stranded RNA molecules. In some embodiments, each strand of the siRNA is about 15-60, 15-50, 15-40 15-30, 15-25, 19-25, 20-30, or 20-24 nucleotides in length. In some embodiments, each strand of the siRNA is 15, 16, 17, 18, 19, 20, 21, 22, 23, 24, 25, 26, 27, 28, 29, 30, 31, 32, 33, 34, or 35 nucleotides in length. In some embodiments, at least one strand of the siRNA has a 3′ overhang of 1-5 nucleotides (e.g., 1, 2, 3, 4, or 5 nucleotides). In some embodiments, siRNA is chemically synthesized. siRNA can also be generated by cleavage of longer dsRNA (e.g., dsRNA greater than 25 nucleotides in length) with Dicer. These enzymes process the dsRNA into biologically active siRNA. In some embodiments, a dsRNA is at least 50 nucleotides to 100, 200, 300, 400, or 500 nucleotides in length. A dsRNA may have a length of 1000, 1500, 2000, 5000 nucleotides, or longer.

In some embodiments, an inhibitory oligonucleotide is an miRNA. In some embodiments, an miRNA is a single-stranded RNA molecule. In some embodiments, an miRNA is a double-stranded RNA molecule. In some embodiments, an miRNA is about 15-60, 15-50, 15-20 40 15-30, 15-25, 19-25, 20-30, or 20-24 nucleotides in length. In some embodiments, an miRNA is 15, 16, 17, 18, 19, 20, 21, 22, 23, 24, 25, 26, 27, 28, 29, 30, 31, 32, 33, 34, or 35 nucleotides in length. In some embodiments, the miRNA is a precursor miRNA (e.g., a premiRNA, or a pri-miRNA). In some embodiments, a precursor miRNA is about 50-150, 60-120, 60-100, or 60-70 nucleotides in length. In some embodiments, a precursor miRNA is at least 50 nucleotides to about 100, 200, 300, 400, or 500 nucleotides in length. A precursor miRNA may be as long as 1000, 1500, 2000, 5000 nucleotides in length, or longer.

In some embodiments, an inhibitory oligonucleotide is an shRNA. A short hairpin RNA or small hairpin RNA (shRNA/Hairpin Vector) is an artificial RNA molecule with a tight hairpin turn that can be used to silence target gene expression via RNA interference (RNAi). Expression of shRNA in cells is typically accomplished by delivery of plasmids or through viral or bacterial vectors. shRNAs are modeled on precursor microRNA (pre-miRNA) and may be cloned into viral vectors where they are transcribed under the control of RNA Polymerase III (Pol II) promoters. shRNAs, in some embodiments, are produced as single-strand molecules of ˜50-70 nucleotides in length, and form stem loop structures with a ˜19-29 base-pair region of double-strand RNA (the stem) bridged by a region of single-strand RNA (the loop) and a short 3′ overhang. Once transcribed, shRNAs exit the nucleus, are cleaved at the loop by the nuclease Dicer in the cytoplasm and enter the RISC to direct cleavage and subsequent degradation of complementary mRNA.

### Vectors

The present disclosure provides engineered vectors comprising the engineered nucleic acids described above. A vector is any nucleic acid that may be used as a vehicle to deliver exogenous (foreign) genetic material to a cell. A vector, in some embodiments, is a DNA sequence that includes an insert (e.g., an inhibitory oligonucleotide) and a larger sequence that serves as the backbone of the vector. Non-limiting examples of vectors include plasmids, viruses/viral vectors, phagemids, cosmids (comprising a plasmid and Lambda phage cos sequences), and artificial chromosomes, any of which may be used as provided herein. In some embodiments, the vector is a viral vector, such as a viral particle. In some embodiments, the vector is an RNA-based vector, such as a self-replicating RNA vector. In some embodiments, a vector also comprises regulatory sequences, such as enhancers and promoters, operably linked to a nucleic acid, such as an inhibitory oligonucleotide.

The vectors, as provided herein, may be used, in some embodiments, to deliver an inhibitory oligonucleotide to a subject or to a cell.

### Methods for Inhibiting Pathogenesis

The present disclosure provides, in some aspects, methods of inhibiting pathogenesis of, for example, a virus, such as a respiratory virus (e.g., an influenza virus or betacoronavirus) in a subject by targeting (e.g., inhibiting) a lncRNA involved in pathogenesis (e.g., pathogen infection (e.g., entry to host cell), propagation, and/or survival).

In one aspect, the disclosure provides a method of inhibiting a viral infection in a subject in need thereof by targeting a lncRNA listed in Table 1 or Table 2. In another aspect, the disclosure provides a method of inhibiting a viral propagation in a subject in need thereof by targeting a lncRNA listed in Table 1 or Table 2. In yet another aspect, the disclosure provides a method of inhibiting a viral survival in a subject in need thereof by targeting a lncRNA listed in Table 1 or Table 2. In some aspects, the disclosure provides a method of reducing viral titer (e.g., by at least 10%, 20%, 30%, 40%, or 50%) in a subject in need thereof by targeting a lncRNA listed in Table 1 or Table 2.

In some embodiments, an inhibitory oligonucleotide of the disclosure targets a lncRNA selected from the group consisting of: DGCR5, AC015987.1, LINC01146, LRRC37A11P, PCAT7, CECR7, MIR503HG, RFPL1S, CYP4A22-AS1, CTC-498J12.1, RP11-360F5.1, LINC00885, LINC00086, GS1-124K5.11, CTD-2127H9.1, AC108488.4, and TMEM44-AS1. In some embodiments, an inhibitory oligonucleotide of the disclosure targets DGCR5. Without wishing to be bound by theory, inhibition of a lncRNAs (e.g., DGCR5) upregulates the type I interferon response pathway.

In some embodiments, a subject is a human subject. In other embodiments, the subject is a livestock animal. The livestock animal may be, for example, a cow, a sheep, a goat, a poultry, or a pig. Other non-human mammals subject to respiratory virus pathogenesis (e.g. infection) are also contemplated herein.

Influenza Infection

In some embodiments, a virus is an influenza virus. Influenza virus infects hosts such as humans and livestock animals (e.g., cattle, sheep, goat, poultry, or pig). Infection can result in global pandemics as the virus spreads among hosts who are contagious but have not yet developed symptoms of infection. Influenza virus primarily infects cells of the airway (e.g., lung epithelial, airway epithelial, and/or alveoli) before spreading throughout the body. The symptoms of influenza virus infection include, for example, congestion, cough, sore throat, fever, chills, aches, and fatigue, and typically appear two days after exposure to the virus and last less than a week. In more severe cases, complications of influenza virus infection can lead to pneumonia, secondary bacterial pneumonia, sinus infection, and worsening of previous health problems including asthma or heart failure. In the most severe cases, influenza virus infection can lead to death, particularly in young children, the elderly, and immunosuppressed subjects. In some embodiments, the present disclosure provides a method of inhibiting the spread of influenza virus in a subject comprising contacting the cells (e.g., airway cells) of the subject with an inhibitory oligonucleotide of the disclosure.

In some embodiments, a subject has been exposed to an influenza virus infection. Exposure to a virus includes indirect or direct contact with the virus. For example, a subject may be considered exposed to influenza virus if the subject was in the presence of another subject who has been infected with the virus. A subject “exposed to” influenza virus may also be “suspected of having” an influenza virus infection. In some embodiments, a subject is infected with (and diagnosed with) an influenza virus infection.

There are four types of influenza viruses: A, B, C and D. Human influenza A and B viruses cause seasonal epidemics of disease almost every winter in the United States. The emergence of a new and very different influenza A virus to infect people can cause an influenza pandemic. Influenza type C infections generally cause a mild respiratory illness and are not thought to cause epidemics. Influenza D viruses primarily affect cattle and are not known to infect or cause illness in people. Influenza A viruses are divided into subtypes based on two proteins on the surface of the virus: the hemagglutinin (H) and the neuraminidase (N). There are 18 different hemagglutinin subtypes and 11 different neuraminidase subtypes (H1 through H18 and N1 through N11 respectively). Influenza A viruses can be further broken down into different strains. Current subtypes of influenza A viruses found in people are influenza A (H1N1) and influenza A (H3N2) viruses. In the spring of 2009, a new influenza A (H1N1) virus (CDC 2009 H1N1 Flu website) emerged to cause illness in people. This virus was very different from the human influenza A (H1N1) viruses circulating at that time. The new virus caused the first influenza pandemic in more than 40 years. That virus (often called “2009 H1N1”) has now replaced the H1N1 virus that was previously circulating in humans. Herein, “H1N1” refers to any H1N1 virus circulating in humans. Influenza B viruses are not divided into subtypes but can be further broken down into lineages and strains. Currently circulating influenza B viruses belong to one of two lineages: B/Yamagata and B/Victoria. See, e.g., cdc.gov/flu/about/viruses/types.htm (Centers for Disease Control and Prevention website).

An influenza virus infection as provided herein may be caused by any strain of influenza virus. In some embodiments, the influenza virus is an influenza type A virus, an influenza type B virus, or an influenza type C virus. In some embodiments, an influenza A strain is selected from the following subtypes: H1N1, H1N2, H1N3, H1N8, H1N9, H2N2, H2N3, H2N8, H3N1, H3N2, H3N8, H4N2, H4N4, H4N6, H4N8, H5N1, H5N2, H5N3, H5N6, H5N8, H5N9, H6N1, H6N2, H6N4, H6N5, H6N6, H6N8, H7N1, H7N2, H7N3, H7N7, H7N8, H7N9, H8N4, H9N1, H9N2, H9N5, H9N8, H10N3, H10N4, H10N7, H10N8, H10N9, H11N2, H11N6, H11N9, H12N1, H12N3, H12N5, H13N6, H13N8, H14N5, H15N2, H15N8, H16N3, H17N10, and H18N11. In some embodiments, the strain of influenza virus is an influenza A (H1N1) strain. In some embodiments, the strain of influenza virus is an influenza A (H3N2) strain. In some embodiments, the strain of influenza virus is an influenza A (H5N1) strain. Non-limiting examples of particular strains of influenza virus include influenza A/WSN/33 (H1N1), influenza A/Hong Kong/8/68 (H3N2), and influenza A/Avian Influenza (H5N1), influenza A/Netherlands/602/2009 (H1N1), and influenza A/Panama/2007/99 (H3N2).

Coronavirus Infection

In some embodiments, a virus is a coronavirus infection. Coronaviruses (CoV) are a large family of zoonotic viruses that are transmitted between animals and people, causing illness ranging from the common cold to more severe diseases such as Middle East Respiratory Syndrome (MERS-CoV) and Severe Acute Respiratory Syndrome (SARS-CoV). Other non-limiting examples of coronaviruses include coronavirus 229E and NL63, which are common human alpha coronaviruses, and OC43 and HKU1, which are common human beta coronaviruses. In some embodiments, the methods and composition provided herein are used to inhibit pathogenesis of an alpha coronavirus. In some embodiments, the methods and composition provided herein are used to inhibit pathogenesis of a beta coronavirus. Several known coronaviruses are circulating in animals that have not yet infected humans.

Common signs of coronavirus infection include respiratory symptoms, fever, cough, shortness of breath, and breathing difficulties. In more severe cases, infection can cause pneumonia, severe acute respiratory syndrome, kidney failure, and even death. On Feb. 11, 2020 the World Health Organization (WHO) announced an official name for the disease that is causing the 2019 novel coronavirus outbreak, first identified in Wuhan City, Hubei Province, China—“coronavirus disease 2019”, abbreviated as “COVID-19.” In COVID-19, ‘CO’ stands for ‘corona,’ ‘VI’ for ‘virus,’ and ‘D’ for disease. Formerly, this disease was referred to as “2019 novel coronavirus” or “2019-nCoV.” In some embodiments, the coronavirus infection being inhibited is COVID-19, also referred to as SARS-CoV2.

In some embodiments, the present disclosure provides a method of inhibiting the spread of coronavirus in a subject comprising contacting the cells (e.g., airway cells) of the subject with an inhibitory oligonucleotide of the disclosure.

In some embodiments, a subject has been exposed to coronavirus. Exposure to a virus includes indirect or direct contact with the virus. For example, a subject may be considered exposed to coronavirus if the subject was in the presence of another subject who has been infected with the virus. A subject “exposed to” coronavirus may also be “suspected of having” a coronavirus infection. In some embodiments, a subject is infected with (and diagnosed with) a coronavirus infection.

### Pharmaceutical Compositions

In some aspects, the present disclosure provides compositions comprising any of the engineered nucleic acids as disclosed herein. In some embodiments, the compositions further comprise a pharmaceutically-acceptable excipient. Non-limiting examples of pharmaceutically-acceptable excipients include water, saline, dextrose, glycerol, ethanol and combinations thereof. The excipient may be selected on the basis of the mode and route of administration, and standard pharmaceutical practice.

Engineered nucleic acids, in some embodiments, may be formulated in a delivery vehicle. Non-limiting examples of delivery vehicles include nanoparticles, such as nanocapsules and nanospheres. See, e.g., Sing, R et al. Exp Mol Pathol. 2009; 86(3):215-223. A nanocapsule is often comprised of a polymeric shell encapsulating a drug (e.g., engineered nucleic acid of the present disclosure). Nanospheres are often comprised of a solid polymeric matrix throughout which the drug (e.g. engineered nucleic acid) is dispersed. In some embodiments, the nanoparticle is a lipid particle, such as a liposome. See, e.g., Puri, A et al. Crit Rev Ther Drug Carrier Syst. 2009; 26(6):523-80. The term ‘nanoparticle’ also encompasses microparticles, such as microcapsules and microspheres.

Methods developed for making particles for delivery of encapsulated agents are described in the literature (for example, please see Doubrow, M., Ed., “Microcapsules and Nanoparticles in Medicine and Pharmacy,” CRC Press, Boca Raton, 1992; Mathiowitz and Langer, J. Controlled Release 5:13-22, 1987; Mathiowitz et al. Reactive Polymers 6:275-283, 1987; Mathiowitz et al. J. Appl. Polymer Sci. 35:755-774, 1988; each of which is incorporated herein by reference).

General considerations in the formulation and/or manufacture of pharmaceutical agents, such as compositions comprising any of the engineered nucleic acids disclosed herein may be found, for example, in Remington's Pharmaceutical Sciences, 18th edition, Mack Publishing Co., Easton, Pa (1990) (incorporated herein by reference in its entirety).

### Methods of Delivery

Any of the engineered nucleic acids or compositions disclosed herein may be administered to a subject (e.g., mammalian subject, such as a human, mouse, rabbit, goat. sheep or pig) to inhibited viral pathogenesis, for example.

Suitable routes of administration include, without limitation, intravenous, intranasal, intramuscular, subcutaneous, and inhalation. In some embodiments, an engineered nucleic acid of the disclosure is administered intravenously, subcutaneous, intramuscularly or intranasally. In some embodiments, an engineered nucleic acid of the disclosure is delivered to the lung. Other routes of administration are contemplated herein. The administration route of an engineered nucleic acid of the disclosure can be changed depending on a number of factors, including the pathogen and/or mechanism of pathogenesis.

In some embodiments, an effective amount of an engineered nucleic acid of the present disclosure is administered to a subject to inhibit pathogenesis of a respiratory virus. A therapeutically effective amount, in some embodiments, is an amount of an inhibitory oligonucleotide (and/or an engineered nucleic acid comprising or encoding the inhibitory oligonucleotide) required to prevent viral infection in a subject. In some embodiments, an effective amount is an amount of inhibitory oligonucleotide required to prevent or reduce viral propagation in a subject. In some embodiments, an effective amount is an amount of inhibitory oligonucleotide required to prevent or reduce viral survival (e.g., length of time a virus survives in a subject). In some embodiments, an effective amount is an amount of inhibitory oligonucleotide required to reduce viral titer in a subject. Effective amounts vary, as recognized by those skilled in the art, depending on the route of administration, excipient usage, and co-usage with other active agents. Effective amounts depend on the subject, including, for example, the weight, sex and age of the subject as well as the strength of the subject's immune system and/or genetic predisposition. Suitable dosage ranges are readily determinable by one skilled in the art. The effective amount (and thus the dosage and/or dosing schedule) of the compositions disclosed herein may also depend on the type of inhibitory oligonucleotide (e.g., DNA, RNA, nucleotide composition, length, etc.).

### EXAMPLES

### Example 1. Screening for lncRNAs that Mediate Influenza Virus Infection

Influenza A virus is a segmented, single-stranded, negative-sense RNA virus member of the Orthomyxoviridae family and a major human pathogen that causes annual epidemics and occasional pandemics with serious public health and economic impact. Influenza infection and replication in host cells is a multi-step process: the virus binds to host surface receptors and enters the cell, then releases its genome into the cytoplasm. The viral genome is subsequently imported to the nucleus, where viral transcription and replication occur, and the new synthesized viral proteins and RNA assemble into progeny viral particles, which release to the extracellular environment by budding. In addition, to establish a productive infection and cause disease, influenza viruses must interact with multiple host cellular factors to support their own replication and to suppress antiviral cell responses.

Identifying the cellular factors involved in viral infection and understanding their roles is critical for exploring the mechanism of viral infection and developing new antiviral therapies. Most investigations to date have focused on the host proteins translated from coding regions of genome, however, the majority (˜98%) of the genome is transcribed as noncoding RNAs, which include a rich subset of long noncoding RNAs (lncRNAs). Importantly, recent advances in the high-throughput sequencing techniques are leading led to the identification of increasing numbers of lncRNAs that are involved in infections and immunological processes; however, the role of cellular lncRNAs in influenza virus infection and pathogenesis remains relatively unexplored.

A CRISPR/Cas9-based genome-wide screening technology was used to identify lncRNAs in host cells that mediate influenza infection, and this provides a new strategy for the discovery and mechanistic studies of influenza-associated lncRNAs. The disclosure is based, in part, on the discovery that knocking out certain lncRNA molecules (e.g., DiGeorge Syndrome Critical Region Gene 5 (DGCR5) lncRNA) inhibits influenza A virus infection in human A549 lung epithelial cells. This is the first time DGCR5 has been identified as a lncRNA related to influenza infection. Without wishing to be bound by any particular theory, exploration of the mechanism of action revealed that knockdown of DGCR5 activates the interferon pathway, which results in up-regulation of type I and II interferons that are known to inhibit viral infection. As the interferon pathway (e.g., IFN-I pathway) is involved in many diseases, modulating DGCR5 lncRNA provides a potential new therapeutic strategy for intervention of these diseases, which include infection of pathogens (viruses, bacteria, fungi, and parasites), cancers, and autoimmune diseases.

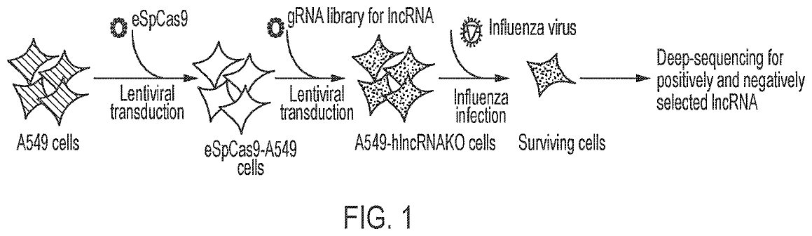

A CRISPR/Cas9-based screening strategy was designed to identify lncRNAs that mediate influenza virus infection, as illustrated in FIG. 1. The exact procedure used was follows: 1) An ‘enhanced specificity’ Streptococcus pyogenes Cas9 (eSpCas9)-expressing A549 stable cell line was established, and A549 cells were transduced with lentivirus expressing the eSpCas9 and blasticidin S deaminase (BSD) genes at MOI=10. The cells were cultured in the presence of 10 μg/mL Blasticidin for 14 days, which killed un-transduced A549 cells and selected for eSpCas9-expressing A549 cells, thereby creating a stable cell line (eSpCas9-A549). 2) An A549-human lncRNA knockout (A549-hlncRNA KO) cell library was also generated. eSpCas9-A549 cells (1.2×10⁷) were transduced with a pool of lentiviruses (MOI=0.4) carrying a paired single-guide RNA (pgRNA) library that containing 12,472 pgRNAs targeting 671 human lncRNAs. This was expected to generate about 4.8×10⁶ transduced cells (A549-hlncRNA KO cells, approximately 384 cells per sgRNA). Transduced cells were selected by being cultured in the presence of 2 ug/mL puromycin for 7-14 days, which allowed for enough time for genome modification by eSpCas9. 3) Selection of influenza virus-resistant A549-hlncRNA KO cells was carried out by infecting A549-hlncRNA KO cells (5×10⁶ cells for each replica) with influenza A/WSN/33 (H1N1) virus (MOI=1.0) and incubating for 2 days to select for cells resistant to virus infection. 4) Deep sequencing was performed to identify relevant lncRNAs in the surviving cells by using PCR to amplify the single guide RNAs (sgRNAs)⁽¹⁾. Deep sequencing also was used to identify enriched lncRNAs, the knockout of which might confer the resistance of cells to influenza infection (Table 2). Uninfected A549-hlncRNA KO cells were used as controls.

Theoretically, cells harboring sgRNAs that knockout lncRNAs associated with resistance to influenza infection, but do not affect cell growth, can survive and expand rapidly. As a consequence, the sgRNAs in these cells should have a high number of reads. By contrast, cells harboring sgRNAs that target lncRNAs that have no effect on resistance to influenza infection or can lead to slow growth even death of cells, will die or grow slowly; thus the sgRNAs in these cells should have no or very few reads. Therefore, a high number of sgRNA reads generally indicates that the knockout of these sgRNA target lncRNAs confers resistance to influenza infection but does not affect cell growth.

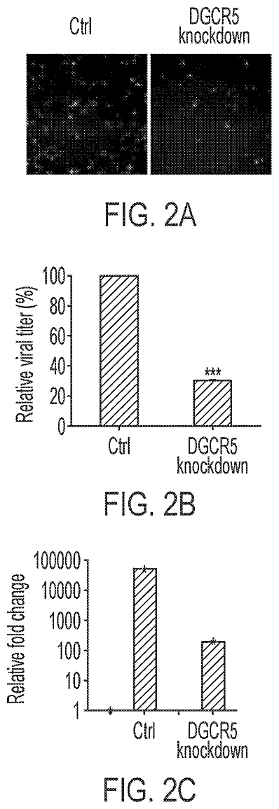

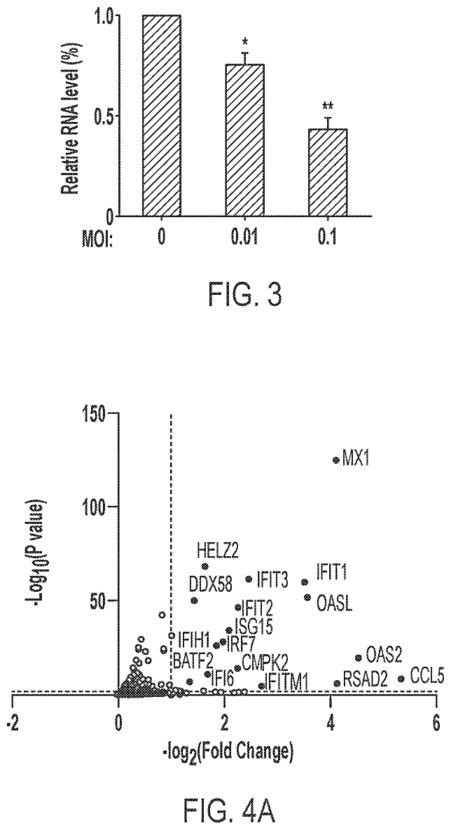

After deep sequencing, enriched lncRNAs (Table 2) were identified using a Model-based Analysis of Genome-wide Crispr/Cas9 Knockout (MAGeCK) method for prioritizing sgRNAs, genes, and pathways in genome-scale Crispr/Cas9 knockout screens⁽²⁾. siRNA technology was then used to validate the top 20 lncRNAs that were enriched in the CRISPR/Cas9-based screening. This analysis resulted in the discovery that multiple lncRNAs produced significant (˜35-80%) inhibition when knocked down with specific siRNAs in A549 cells (Table 1). The most enriched lncRNA (DGCR5) was also the most potent in that it suppressed influenza infection by ˜80% in A549 cells (Table 1 & FIGS. 2A-2B). Importantly, when the same experiment was carried out in the influenza infected human Lung Airway Chip, which more closely mimics human lung airway pathophysiology^{(3, 4)}, treatment with DGCR5 lncRNA inhibited infection by ˜100-fold (FIG. 2C). In addition, it was found that infection of A549 cells with influenza virus resulted in a significant decrease in DGCR5 lncRNA levels, which may contribute to its infectivity (FIG. 3).

A549 cells were transfected with siRNAs (IDT lnc) to knockdown target lncRNA. Twenty-four (24) hours later, cells were infected with GFP-labeled influenza A/PR8/34 (H1N1) virus (MOI=0.01). GFP signals were recorded 48 hours post-infection. Scramble siRNAs were used as control. The inhibition rate=(1−GFP-positive cell number in tested group/GFP-positive cell number in control group)×100%.

TABLE 1
The inhibition rate of selected IncRNAs
against influenza infection.

| | | IncRNA | Example | SEQ |
|---|---|---|---|---|
| | Inhibition | Ensemble | gRNA | ID |
| IncRNA | rate (%) | ID | Sequences | NOs: |
| Control | 0 | — | — | |
| DGCR5 | 77 | ENST00000440005 | CCGCCCCTCCTAGCCAGCTG | 1 |
| | | | GAGGCAGTGATAGATGATGG | 2 |
| | | | CGGGTACCGAGAGTAGGTGG | 3 |
| | | | ATCAGGACCAGCTCGGGCAG | 4 |
| | | | GTGCCTCTTGGCTCTCCAGT | 5 |
| | | | GGCGCCTGGATGCCGGCCCG | 6 |
| | | | GAGGTAACAGAGTGGCCCCG | 7 |
| | | | GCCCAGACATCCGCAGCCCG | 8 |
| | | | CCCCATCTTACTGCAAGGCC | 9 |
| | | | CCCCATCTTACTGCAAGGCC | 10 |
| | | | CCCCATCTTACTGCAAGGCC | 11 |
| | | | CTGGGTGTGAGGTCCCGCAG | 12 |
| | | | ATCAGGACCAGCTCGGGCAG | 13 |
| | | | ATCAGGACCAGCTCGGGCAG | 14 |
| | | | CCCCATCTTACTGCAAGGCC | 15 |
| | | | CCCCATCTTACTGCAAGGCC | 16 |
| AC015987.1 | 10 | ENST00000419211 | TATTTCCCTACAATATCGCA | 17 |
| | | | ATGAGCCCACAACTGGGGGA | 18 |
| | | | ATATGTGACTGTCCTTGAAG | 19 |
| | | | GAGAACTTTAATTTACCAAG | 20 |
| LINC01146 | −6 | ENST00000556673 | GGAGATCAATGGGCAGGAGC | 21 |
| | | | GAAGATATTTCCATGTCTGC | 22 |
| | | | CCTGGATTCTAGATCATAGG | 23 |
| | | | GAGATGACCACTTAGACCAC | 24 |
| LRRC37A11P | 46 | ENST00000425901 | CCGCCCCCACCGGCAGACCC | 25 |
| | | | TACAGGTGTCATAAAGGACG | 26 |
| | | | TGTGGGATAAGATCGCGGAG | 27 |
| | | | GCCCCCACCGGCAGACCCCG | 28 |
| | | | CAGCGTTACAGTGAAACCTG | 29 |
| | | | GCGCTAACTCCAGAGCCAGA | 30 |
| | | | CTGGGAGGTTGAACATTCTG | 31 |
| | | | GTCCATGCCAACACGAGGCC | 32 |
| | | | ACCATCTCTTAACTCAGCAG | 33 |
| | | | CCTCAGGTTGGCAACTACTG | 34 |
| | | | CTGAGTATTCTACAACCCCA | 35 |
| | | | GTCCATGCCAACACGAGGCC | 36 |
| | | | GTCCATGCCAACACGAGGCC | 37 |
| | | | GCTTCCAGAGACACCTACAG | 38 |
| | | | GCTTCCAGAGACACCTACAG | 39 |
| | | | GCTTCCAGAGACACCTACAG | 40 |
| | | | ACCATCTCTTAACTCAGCAG | 41 |
| | | | CCCGGGGCCAGAGCAGTTAG | 42 |
| | | | CCCGGGGCCAGAGCAGTTAG | 43 |
| | | | CTGAGTATTCTACAACCCCA | 44 |
| LINC00176 | 41 | ENST00000444463 | GTCGTTGAGGCCAGCGGGCG | 45 |
| | | | GCGACAGCAGCCAAGGATCG | 46 |
| | | | GAGCCAGCTGGTTCTTGTGT | 47 |
| | | | GCTGGTTCTTGTGTAGGCCG | 48 |
| | | | TCACAGAGGAGGCACTGTGG | 49 |
| | | | CCTGGGGTCTGAGTGCAAAC | 50 |
| | | | CAGTCGTTGAGGCCAGCGGG | 51 |
| | | | CCATGTCTTGGGACCTGCTG | 52 |
| | | | GTTGAGGCCAGCGGGCGGGG | 53 |
| | | | GACATGGTCAATCCCGGGCA | 54 |
| | | | AGCCCTGGATCCCCTGGGAG | 55 |
| | | | CAGTCGTTGAGGCCAGCGGG | 56 |
| | | | ATCCCTGCCTGCACGTGGGG | 57 |
| | | | GTTCAGGGGATACTTCCTGC | 58 |
| | | | TCATGTGCGTGGCCTGCTGG | 59 |
| | | | TCATGTGCGTGGCCTGCTGG | 60 |
| | | | CTCCTGGGTATCTTTACGGC | 61 |
| | | | GTGACAGTGTCACCAGTAAG | 62 |
| | | | CTCCTGGGTATCTTTACGGC | 63 |
| | | | GTGACAGTGTCACCAGTAAG | 64 |
| PCAT7 | 43 | ENST00000452148 | AGTGGTAGGAGGGCACCCGT | 65 |
| | | | TAACTGTGACTCATGAGAGA | 66 |
| | | | TACTGTCCCTACTGTAAAGG | 67 |
| | | | ACTGGCATCGGCGCTAGCGT | 68 |
| | | | TGCCAAGCCATGCTAGACTG | 69 |
| | | | GTATGTCAATGTTTAAACTG | 70 |
| | | | GGCGCTAGCGTTGGTAGCAG | 71 |
| | | | CTGAATATCTATGGACCTGG | 72 |
| | | | TGCCAAGCCATGCTAGACTG | 73 |
| | | | CTGAATATCTATGGACCTGG | 74 |
| | | | TGCCAAGCCATGCTAGACTG | 75 |
| | | | CTGTCAACCCACATAATTGG | 76 |
| | | | AGAACTCCATCCTTCCATGG | 77 |
| | | | TGCCAAGCCATGCTAGACTG | 78 |
| | | | AAATTATGTTAAGACTGGGG | 79 |
| | | | AGAACTCCATCCTTCCATGG | 80 |
| CECR7 | 40 | ENST00000609932 | TGGTGAAGCTGCATGGGCCA | 81 |
| | | | GACAAAACAAGTCTCAATGG | 82 |
| | | | AGGGAGGTGCACCAAAACCT | 83 |
| | | | CGAGACAGGGGAACCCCGAG | 84 |
| | | | GGACATTGTAGGTGCTCCCA | 85 |
| | | | GACCTCTGCCCTCTGTGCCA | 86 |
| | | | ATAGGAGCGCCCTGGCCCAG | 87 |
| | | | GTCTGGGCAAGCAAGATCCC | 88 |
| | | | GGGCCGCATGCGGACCCTAG | 89 |
| | | | CGAGACAGGGGAACCCCGAG | 90 |
| | | | GGATCCGCACAGCGGCCCTG | 91 |
| | | | ATAGGAGCGCCCTGGCCCAG | 92 |
| | | | TAGGGTCCGCATGCGGCCCG | 93 |
| | | | GGGCCGCATGCGGACCCTAG | 94 |
| | | | GTGCAGAGGTCACGAGACAG | 95 |
| | | | GGATCCGCACAGCGGCCCTG | 96 |
| MIR503HG | 39 | ENST00000440570 | GTGGATAGAGGAGCGGGTAG | 97 |
| | | | GAATCAATGAAAAGATCAGG | 98 |
| | | | ACTGGCTCTCCAATTTGCTG | 99 |
| | | | ACTGGCTCTCCAATTTGCTG | 100 |
| RFPLIS | 18 | ENST00000461286 | GAAAGAGCAAACCTGAAGGG | 101 |
| | | | GCAGCAGTGAATATGTGCAC | 102 |
| | | | TCGCATGGTGAATCATGTCA | 103 |
| | | | GAGAGTGGCTCTTGCACCTG | 104 |
| CYP4A22-AS1 | 12 | ENST00000444042 | TTGGAAGACGATGGCATCAG | 105 |
| | | | TGTGCTGGAGGTGTCACAGT | 106 |
| | | | CAGTTTATTCCACACCGCTG | 107 |
| | | | CGCTGCACAGCCAGTCCCAG | 108 |
| | | | TTGCAGGCTGCCTCAAGCCC | 109 |
| | | | GAACAGGCTTTCCTTGATGG | 110 |
| | | | CTGGTGGCTGCACGCTCCAA | 111 |
| | | | GTACCAGCCTGGATCTCTTG | 112 |
| | | | GACAGGCTGAGCTAGTGAGC | 113 |
| | | | AACTCCAGCCATCTGTCCCG | 114 |
| | | | GGTGGAGTTCTGAAACAGCT | 115 |
| | | | GGTGAAGAGCCCCGAAGGAA | 116 |
| | | | GACAGGCTGAGCTAGTGAGC | 117 |
| | | | GCTTCCTTGATGTCAACCCT | 118 |
| | | | GTACCAGCCTGGATCTCTTG | 119 |
| | | | GGTGGAGTTCTGAAACAGCT | 120 |
| | | | TGTGCTGGAGGTGTCACAGT | 121 |
| | | | GAACAGGCTTTCCTTGATGG | 122 |
| | | | GGCTTCGGCTTAATGAACTG | 123 |
| | | | GGCTTCGGCTTAATGAACTG | 124 |
| | | | GGTGGAGTTCTGAAACAGCT | 125 |
| | | | CGCTGCACAGCCAGTCCCAG | 126 |
| CTC-498J12.1 | 48 | ENST00000479830 | ATCCACCCCGAATTTAGGCA | 127 |
| | | | GGGTACACGCAATGGAGGCA | 128 |
| | | | GAACCTGAATGACTTAGGGA | 129 |
| | | | AATGAATGCGGCTTGAGTAG | 130 |
| | | | TACAGAAACGATGTAGTTGG | 131 |
| | | | TCCTAATCAGCAGAAAAGGG | 132 |
| | | | CAACTTGGATAGGAATAAGA | 133 |
| | | | ACCCCACCATGTTTAATTAG | 134 |
| | | | GAGCTAGGGCATACTTTAGG | 135 |
| | | | GGCCAGTCTTGCTTTCTGGG | 136 |
| | | | TTGGATAGGAATAAGACGGA | 137 |
| | | | ATAAAGGGTACACGCAATGG | 138 |
| | | | ACTCAAAGTTGATATTAAAG | 139 |
| | | | CAACTTGGATAGGAATAAGA | 140 |
| | | | GGGTACACGCAATGGAGGCA | 141 |
| | | | CAACTTGGATAGGAATAAGA | 142 |
| | | | TTGAGTTAGCAAGTAACTAT | 143 |
| | | | GGCCAGTCTTGCTTTCTGGG | 144 |
| | | | TTCAGATCAGATTAGTACAG | 145 |
| | | | TCCTAATCAGCAGAAAAGGG | 146 |
| | | | TCCTAATCAGCAGAAAAGGG | 147 |
| | | | TTGAGTTAGCAAGTAACTAT | 148 |
| RP11-360F5.1 | 19 | ENST00000509449 | TGCTCGTGGAATAACACAAA | 149 |
| | | | CTTAAACAAGGTTGGGAGGG | 150 |
| | | | AATGGAACCAGCTTGAACCT | 151 |
| | | | GTGGAATAACACAAAGGGCC | 152 |
| | | | CGCAGTGTGAGAGGAGCCTA | 153 |
| | | | CAATACGGCATTTCTTCAAG | 154 |
| | | | AATGGAACCAGCTTGAACCT | 155 |
| | | | CAATGGGGTAACAACTCCTG | 156 |
| | | | CAATACGGCATTTCTTCAAG | 157 |
| | | | AATGGAACCAGCTTGAACCT | 158 |
| LINC00885 | 30 | ENST00000457079 | GCATCTCACCCCCTTAACCC | 159 |
| | | | GCCGCCGGGGTAGGTTCGAG | 160 |
| | | | CCTCTCAGCACTCGCTACCG | 161 |
| | | | ACTACTTCATCCCTCTGGGC | 162 |
| | | | GCTGATGATTCATGGTGCGC | 163 |
| | | | ACAACACGTGACCCCGGAGA | 164 |
| | | | TGCAATGACAGCCCAGAGCG | 165 |
| | | | AGGAGGGCAACGAGGCCGGC | 166 |
| | | | GGCTACATTGCTCCCCAGAG | 167 |
| | | | GGCTACATTGCTCCCCAGAG | 168 |
| | | | GGCTACATTGCTCCCCAGAG | 169 |
| | | | GGCTACATTGCTCCCCAGAG | 170 |
| | | | GGCTACATTGCTCCCCAGAG | 171 |
| | | | GGCTACATTGCTCCCCAGAG | 172 |
| | | | GGCTACATTGCTCCCCAGAG | 173 |
| | | | GGCTACATTGCTCCCCAGAG | 174 |
| LINC00086 | 5 | ENST00000417443 | CCTCGTGGCCGCTCAGGCCC | 175 |
| | | | GGTCCTGCCTTCAGGCTCCG | 176 |
| | | | CTCCGGAGGCGCCCGGAAAG | 177 |
| | | | CGGAGGCGCCCGGAAAGAGG | 178 |
| GS1-124K5.11 | 48 | ENST00000449307 | ACAGCCCTTGGTGGACATGG | 179 |
| | | | TCTTATGTAGCATTGTGGAA | 180 |
| | | | CGGGGGGGTGAACTATCTGT | 181 |
| | | | CGGGGGGGTGAACTATCTGT | 182 |
| CTD-2127H9.1 | 27 | ENST00000513480 | GATGGATATGTACAATCAAG | 183 |
| | | | GCGATTCAGAAATAAAGCTT | 184 |
| | | | TTGTGTAGGTGATCAAGCCA | 185 |
| | | | GGTCTTATAAGAGTGGGTGA | 186 |
| RP11-475N22.4 | −13 | ENST00000468377 | GGCTAAATCCTCCAATTGGG | 187 |
| | | | ATACCAGAGTTGTCATGAGG | 188 |
| | | | GGATGAGCTCCCTCCCCAAG | 189 |
| | | | GAATGAGGACTAGAGGGGGC | 190 |
| | | | TGTGTGACAGCTCAGGCCAG | 191 |
| | | | AGTCCCTCCTGCCCACAAGG | 192 |
| | | | ACATGGATGCTACCAACCAC | 193 |
| | | | GTCAGGCCAAGCATGCAGGC | 194 |
| | | | ATCAGCATGGATGCCTGCAA | 195 |
| | | | AGGGGCTGGTGCTCTGCCGC | 196 |
| | | | ATCAGCATGGATGCCTGCAA | 197 |
| | | | TGAGTCGATCCTGCGCACAG | 198 |
| | | | ACAACCTGTCCCCACACGTG | 199 |
| | | | CATGGAAATACCAGGTGCTG | 200 |
| | | | CATGGAAATACCAGGTGCTG | 201 |
| | | | ACAACCTGTCCCCACACGTG | 202 |
| AC108488.4 | 35 | ENST00000422961 | TGTTGGTGCTGAGTGCGGAT | 203 |
| | | | CTCAGTTCGTAAACACTCAG | 204 |
| | | | TGTTCGTGCCGCACAAATAG | 205 |
| | | | TCTCTAATGTGTAAAGTAAG | 206 |
| | | | TACCTGATTCATGTTACAGC | 207 |
| | | | GGGAAGCCGCCAGTATATGG | 208 |
| | | | GTCCGGGAAGGTCTTTGTCA | 209 |
| | | | GGTGGACGCAAGCTTGGTCC | 210 |
| | | | GGGCACTGTTAAGTGGAAAA | 211 |
| | | | AGCTTGGTCCCAGAGGGGAG | 212 |
| | | | GTCCGGGAAGGTCTTTGTCA | 213 |
| | | | GGACTCAAGCTTGGTCCCAG | 214 |
| | | | AGCTTGGTCCCAGAGGGGAG | 215 |
| | | | GGTGGACACATGCTTAGCTG | 216 |
| | | | GGACTCAAGCTTGGTCCCAG | 217 |
| | | | GGTGGACGCAAGCTTGGTCC | 218 |
| | | | AGTGCATGAAGCCGGAGGGA | 219 |
| | | | GGTGGACACATGCTTAGCTG | 220 |
| | | | GGACTCAAGCTTGGTCCCAG | 221 |
| | | | ACTCAGGGGTAACATGGGGA | 222 |
| TMEM44-AS1 | 40 | ENST00000453671 | CTAGCTAGGAAACTGCGGAG | 223 |
| | | | TATCTTATCTCAATAGGAAA | 224 |
| | | | GGACTCTCGCTCCTCGCGGG | 225 |
| | | | GTGGAGACCCCATCTCATGA | 226 |
| | | | AGTAGGGCGCAAGATGTCGG | 227 |
| | | | TCAAGTCCGGAGCAGAGGCG | 228 |
| | | | AGAATTCATGCTGTGAGAAC | 229 |
| | | | GAGGCACGCCACACAACCCT | 230 |
| | | | TCCATTTCTCAATACGCCAG | 231 |
| | | | TTATCTCCTTAAAAACCGAG | 232 |
| | | | CAGCCGATGGAAAATTCAGA | 233 |
| | | | GTAGTTCACTGTGGATGAAG | 234 |
| | | | CAGCCGATGGAAAATTCAGA | 235 |
| | | | TCTCTTCATCATACTGAGGG | 236 |
| | | | AGTAGGGCGCAAGATGTCGG | 237 |
| | | | CACGTGTTCTGCTCAAAAGA | 238 |
| | | | TCCATAGTTGGAAAATGTGG | 239 |
| | | | CTACGAACACTGGCAGCCGA | 240 |
| | | | TCCATAGTTGGAAAATGTGG | 241 |
| | | | GTAGTTCACTGTGGATGAAG | 242 |
| | | | CAGCCGATGGAAAATTCAGA | 243 |
| | | | GGGCCGAGTGCTGGCTGCCC | 244 |

TABLE 2
Enriched IncRNAs

| IncRNA | IncRNA Ensemble ID | Enrichment score |
|---|---|---|
| DGCR5 | ENST00000440005 | 1.900990099 |
| AC015987.1 | ENST00000419211 | 1.825082509 |
| LINC01146 | ENST00000556673 | 1.429042904 |
| AR | ENST00000374690, | 1.363036303 |
| | ENST00000396043 | |
| LRRC37A11P | ENST00000425901 | 1.326732673 |
| RPL36 | ENST00000347512, | 1.320132013 |
| | ENST00000394580 | |
| AAVS1 | — | 1.227722772 |
| LINC00176 | ENST00000444463 | 1.095709571 |
| FOXA1 | ENST00000250448 | 1.089108912 |
| PCAT7 | ENST00000452148 | 1.07590759 |
| CECR7 | ENST00000609932, | 1.06270627 |
| | ENST00000441006 | |
| RSL24D1 | ENST00000260443 | 1.036303631 |
| MIR503HG | ENST00000440570, | 1.02640264 |
| | ENST00000362227 | |
| RFPL1S | ENST00000461286 | 1.00990099 |
| CYP4A22-AS1 | ENST00000444042 | 0.99339934 |
| RP5-1073O3.2 | ENST00000429398 | 0.95709571 |
| TPT1-AS1 | ENST00000517509, | 0.947194719 |
| | ENST00000524062 | |
| RP11-548L20.1 | ENST00000514877 | 0.907590759 |
| LINC01060 | ENST00000510005, | 0.891089109 |
| | ENST00000503580 | |
| RP1-122P22.2 | ENST00000412571 | 0.887788779 |
| AC093375.1 | ENST00000448255 | 0.884488449 |
| LINC00844 | ENST00000432535 | 0.871287129 |
| CCDC183-AS1 | ENST00000414656 | 0.864686469 |
| RP11-734K21.5 | ENST00000565044 | 0.854785479 |
| AC104135.2 | ENST00000418001 | 0.851485149 |
| CTC-527H23.3 | ENST00000561876 | 0.848184818 |
| H19 | ENST00000390168, | 0.844884489 |
| | ENST00000431095 | |
| ANKRD18CP | ENST00000354752 | 0.844884488 |
| RP11-70F11.8 | ENST00000546821 | 0.841584158 |
| RP11-167H9.6 | ENST00000472890, | 0.834983498 |
| | ENST00000498005 | |
| RP6-65G23.3 | ENST00000554032 | 0.811881188 |
| RAP2C-AS1 | ENST00000441399 | 0.805280528 |
| RP11-128M1.1 | ENST00000447956 | 0.801980198 |
| RP11-76N22.2 | ENST00000458097 | 0.798679868 |
| RPL21 | ENST00000311549 | 0.788778878 |
| LINC00639 | ENST00000553932, | 0.788778878 |
| | ENST00000554732 | |
| LINC00657 | ENST00000565493 | 0.765676568 |
| CTD-2541M15.1 | ENST00000522897 | 0.765676568 |
| LINC01087 | ENST00000431979 | 0.726072607 |
| MAPKAPK5-AS1 | ENST00000456429 | 0.712871287 |
| RP11-195M16.1 | ENST00000415166 | 0.709570957 |
| AC005329.7 | ENST00000501448 | 0.693069307 |
| CSAG4 | ENST00000361201 | 0.689768977 |
| RP11-760H22.2 | ENST00000520544 | 0.676567657 |
| RP1-179N16.6 | ENST00000526611 | 0.669966997 |
| RP11-333I13.1 | ENST00000568862 | 0.646864686 |
| RP11-435O5.2 | ENST00000433644 | 0.617161716 |
| AC084809.2 | ENST00000435733 | 0.613861386 |
| CTD-2566J3.1 | ENST00000554679 | 0.600660066 |
| AC009478.1 | ENST00000429816 | 0.551155116 |
| CTB-181F24.1 | ENST00000521341 | 0.547854785 |
| RP11-308D16.4 | ENST00000431464 | 0.541254125 |
| RP11-314C16.1 | ENST00000429060 | 0.534653465 |
| AC020571.3 | ENST00000433933 | 0.528052805 |
| RP11-725D20.1 | ENST00000504537 | 0.514851485 |
| RP11-367G18.1 | ENST00000427157 | 0.501650165 |
| LINC01132 | ENST00000437601 | 0.498349835 |
| HOXB13 | ENST00000290295 | 0.488448845 |
| RP11-462P6.1 | ENST00000558245 | 0.488448845 |
| RP5-1142A6.9 | ENST00000564984 | 0.485148515 |
| FTX | ENST00000603672 | 0.481848185 |
| LINC00471 | ENST00000313064 | 0.478547855 |
| RP11-498P14.5 | ENST00000607322 | 0.468646865 |
| RP11-318M2.2 | ENST00000500902, | 0.465346535 |
| | ENST00000499522 | |
| CTD-2587M2.1 | ENST00000512693 | 0.452145215 |
| RP11-304F15.7 | ENST00000574365 | 0.445544555 |
| DLGAP1-AS2 | ENST00000572856 | 0.435643564 |
| RP11-299G20.2 | ENST00000558838 | 0.429042904 |
| RP11-789C1.1 | ENST00000504509 | 0.422442244 |
| RPL14 | ENST00000338970, | 0.349834983 |
| | ENST00000396203 | |
| RP11-151A6.4 | ENST00000454752 | 0.339933993 |
| RP11-627G23.1 | ENST00000533390 | 0.333333333 |
| CTD-2016O11.1 | ENST00000509924 | 0.333333333 |
| ENTPD1-AS1 | ENST00000416301 | 0.323432343 |
| AE000661.37 | ENST00000514473 | 0.316831683 |
| RP11-134G8.8 | ENST00000430471 | 0.306930693 |
| SNHG5 | ENST00000427501, | 0.300330033 |
| | ENST00000589187 | |
| EZH2 | ENST00000320356, | 0.293729373 |
| | ENST00000350995 | |
| RPL37A | ENST00000491306 | 0.280528053 |
| CTD-3051D23.4 | ENST00000553344 | 0.273927393 |
| LINC00925 | ENST00000538734, | 0.267326733 |
| | ENST00000536780 | |
| RP11-732M18.3 | ENST00000432358 | 0.260726073 |
| JRK | ENST00000591357 | 0.240924092 |
| RP11-802E16.3 | ENST00000529934 | 0.237623762 |
| LINC00984 | ENST00000560415 | 0.231023102 |
| EGOT | ENST00000414938 | 0.224422442 |
| RPL39 | ENST00000361575 | 0.224422442 |
| RP11-473M20.14 | ENST00000575139 | 0.217821782 |
| TGGENE | — | 0.211221122 |
| RP11-15I11.2 | ENST00000444750 | 0.198019802 |
| RP11-677M14.3 | ENST00000504932 | 0.184818482 |
| RP11-170M17.1 | ENST00000444770 | 0.181518152 |
| RP11-65J3.1 | ENST00000423122 | 0.168316832 |
| RP11-97O12.7 | ENST00000561140 | 0.168316832 |
| SNAI3-AS1 | ENST00000563475 | 0.165016502 |
| AC095067.1 | ENST00000429010 | 0.161716172 |
| LINC01133 | ENST00000423943 | 0.155115512 |
| RP11-540A21.2 | ENST00000500698 | 0.148514851 |
| RP1-261D10.2 | ENST00000555771 | 0.145214522 |
| RP11-268G12.1 | ENST00000422048 | 0.118811881 |
| RP11-90K6.1 | ENST00000498032 | 0.099009901 |
| RP11-373N22.3 | ENST00000501695 | 0.095709571 |
| RP11-394O4.3 | ENST00000521756 | 0.079207921 |
| LINC00205 | ENST00000433465 | 0.075907591 |
| RP11-399D6.2 | ENST00000436786 | 0.072607261 |
| RP11-400K9.4 | ENST00000426023 | 0.069306931 |
| RP11-96D1.7 | ENST00000563175 | 0.062706271 |
| KB-1460A1.1 | ENST00000524369 | 0.046204621 |
| LINC00277 | ENST00000415504, | 0.04620462 |
| | ENST00000440444 | |
| RP11-269F19.2 | ENST00000428791 | 0.04290429 |
| RPL13 | ENST00000393099, | 0.02970297 |
| | ENST00000311528 | |
| RPL23 | ENST00000479035 | 0.01650165 |
| RPL37 | ENST00000274242 | 0.00990099 |
| LINC00565 | ENST00000562710 | 0 |
| LINC00174 | ENST00000421767 | −0.00990099 |
| RP11-353N14.2 | ENST00000576963 | −0.01320132 |
| CTD-2228K2.7 | ENST00000607286 | −0.01650165 |
| AC004463.6 | ENST00000565162 | −0.01980198 |
| AC097468.4 | ENST00000441450 | −0.0330033 |
| RP1-90G24.10 | ENST00000434942 | −0.03630363 |
| RP11-46A10.5 | ENST00000358073 | −0.052805281 |
| RP11-401P9.4 | ENST00000563424 | −0.05940594 |
| RP11-244F12.3 | ENST00000561241 | −0.085808581 |
| RP11-680A11.5 | ENST00000550263 | −0.092409241 |
| TLR8-AS1 | ENST00000451564 | −0.115511551 |
| AC011747.7 | ENST00000412712, | −0.158415842 |
| | ENST00000455965 | |
| RP11-16P6.1 | ENST00000568928 | −0.194719472 |
| AC008268.1 | ENST00000425887 | −0.207920792 |
| RP11-148K1.12 | ENST00000485974 | −0.214521452 |
| RP11-21A7A.3 | ENST00000543817 | −0.234323432 |
| RP5-1086K13.1 | ENST00000456414 | −0.247524752 |
| TTTY14 | ENST00000454875, | −0.250825083 |
| | ENST00000324446 | |
| TSTD3 | ENST00000452647 | −0.254125413 |
| RP11-158M2.4 | ENST00000558637 | −0.267326733 |
| RP11-148B18.3 | ENST00000418242 | −0.277227723 |
| RP11-295G20.2 | ENST00000416221 | −0.280528053 |
| RP11-566E18.3 | ENST00000562038 | −0.290429043 |
| MMP24-AS1 | ENST00000566203 | −0.303630363 |
| AC007405.6 | ENST00000426475 | −0.320132013 |
| RP11-37C7.3 | ENST00000564211 | −0.330033003 |
| RP5-837M10.4 | ENST00000432210 | −0.333333334 |
| LMCD1-AS1 | ENST00000446281, | −0.336633663 |
| | ENST00000441861 | |
| NFE4 | ENST00000420058 | −0.343234323 |
| ABCC5-AS1 | ENST00000422946 | −0.353135313 |
| RPL18A | ENST00000222247 | −0.356435644 |
| HOTAIR | ENST00000424518 | −0.359735974 |
| RP11-193H5.1 | ENST00000450451 | −0.366336634 |
| AC100830.3 | ENST00000560387 | −0.369636964 |
| RP11-996F15.2 | ENST00000553105 | −0.372937294 |
| RP11-279F6.1 | ENST00000498938, | −0.376237624 |
| | ENST00000560882 | |
| TTTY15 | ENST00000440408 | −0.379537954 |
| RP11-983P16.2 | ENST00000435621 | −0.382838284 |
| ISM1-AS1 | ENST00000431407 | −0.386138614 |
| RP3-405J10.3 | ENST00000552061 | −0.392739274 |
| RP11-203B7.2 | ENST00000570186 | −0.402640264 |
| RP11-439E19.10 | ENST00000567832 | −0.405940594 |
| LINC01090 | ENST00000434418 | −0.415841584 |
| H1FX-AS1 | ENST00000433902 | −0.419141914 |
| AC017074.2 | ENST00000424612 | −0.422442244 |
| LINC00839 | ENST00000429940 | −0.425742574 |
| LINC01091 | ENST00000515769, | −0.455445545 |
| | ENST00000508111 | |
| TMEM161B-AS1 | ENST00000501869, | −0.462046205 |
| | ENST00000510087 | |
| NEAT1 | ENST00000384994, | −0.475247524 |
| | ENST00000501122 | |
| SPTY2D1-AS1 | ENST00000501599 | −0.475247525 |
| RP11-317N8.5 | ENST00000555918 | −0.488448845 |
| RP1-278O22.1 | ENST00000417299 | −0.504950495 |
| AC092835.2 | ENST00000425953 | −0.518151815 |
| RP11-711M9.1 | ENST00000496886 | −0.524752476 |
| RP11-356J5.12 | ENST00000504610 | −0.541254125 |
| RP4-816N1.7 | ENST00000547042 | −0.561056106 |
| AC012074.2 | ENST00000431557 | −0.564356436 |
| RP11-531A24.5 | ENST00000517664 | −0.574257426 |
| NDUFB2-AS1 | ENST00000465466 | −0.577557756 |
| RP11-157P1.4 | ENST00000414042 | −0.590759076 |
| AC109333.10 | ENST00000438266 | −0.607260726 |
| GS1-421I3.2 | ENST00000454625 | −0.610561056 |
| RP11-18H7.1 | ENST00000484076 | −0.633663366 |
| CTB-113P19.4 | ENST00000564471 | −0.636963696 |
| SNHG7 | ENST00000414282 | −0.640264026 |
| NOP14-AS1 | ENST00000503709 | −0.640264026 |
| RP11-179B15.5 | ENST00000440189 | −0.650165017 |
| PCA4 | — | −0.663366337 |
| CTD-2020K17.3 | ENST00000587534 | −0.666666667 |
| AC002456.2 | ENST00000412669 | −0.683168317 |
| OSGEPL1-AS1 | ENST00000521819 | −0.686468647 |
| AC097724.3 | ENST00000452212 | −0.706270627 |
| AC012370.2 | ENST00000419244 | −0.716171617 |
| LINC00624 | ENST00000437831 | −0.722772277 |
| DANCR | ENST00000444958 | −0.739273927 |
| AC074289.1 | ENST00000438115 | −0.742574257 |
| RP11-353N14.4 | ENST00000572353 | −0.768976898 |
| RP11-142C4.6 | ENST00000533954 | −0.772277228 |
| HOXA-AS2 | ENST00000518088 | −0.775577558 |
| LINC01024 | ENST00000499203, | −0.782178218 |
| | ENST00000523452 | |
| CTA-250D10.23 | ENST00000566575 | −0.785478548 |
| RP11-539L10.3 | ENST00000515205 | −0.792079208 |
| CTD-2001C12.1 | ENST00000499025 | −0.795379538 |
| GHRLOS | ENST00000439539 | −0.808580858 |
| RP4-798A10.2 | ENST00000457898 | −0.818481848 |
| Z83851.3 | ENST00000415205, | −0.821782178 |
| | ENST00000332965 | |
| VIM-AS1 | ENST00000605833, | −0.825082508 |
| | ENST00000437232 | |
| RP11-47P18.1 | ENST00000482003 | −0.858085809 |
| RP11-382A20.2 | ENST00000565513 | −0.867986799 |
| NBPF18P | ENST00000432386 | −0.881188119 |
| RP11-53O19.1 | ENST00000514597 | −0.894389439 |
| RP11-473M20.16 | ENST00000570843 | −0.904290429 |
| RP11-540O11.1 | ENST00000503052 | −0.910891089 |
| CTC-498J12.1 | ENST00000479830 | −0.914191419 |
| RP11-360F5.1 | ENST00000509449 | −0.947194719 |
| LINC00261 | ENST00000564492 | −0.950495049 |
| LINC00885 | ENST00000457079 | −0.95049505 |
| LINC00086 | ENST00000417443 | −0.96039604 |
| GS1-124K5.11 | ENST00000449307 | −0.98349835 |
| CTD-2127H9.1 | ENST00000513480 | −0.99669967 |
| RPL3 | ENST00000216146 | −1.07590759 |
| RP11-475N22.4 | ENST00000468377 | −1.125412542 |
| AC108488.4 | ENST00000422961 | −1.313531352 |
| RPS3A | ENST00000274065 | −1.623762377 |
| TMEM44-AS1 | ENST00000453671 | −1.716171617 |
| RPL34 | ENST00000394667, | −1.726072607 |
| | ENST00000394668 | |

### LncRNA DGCR5 Negatively Regulates Type I Interferon Pathway Via Modulating IRF3

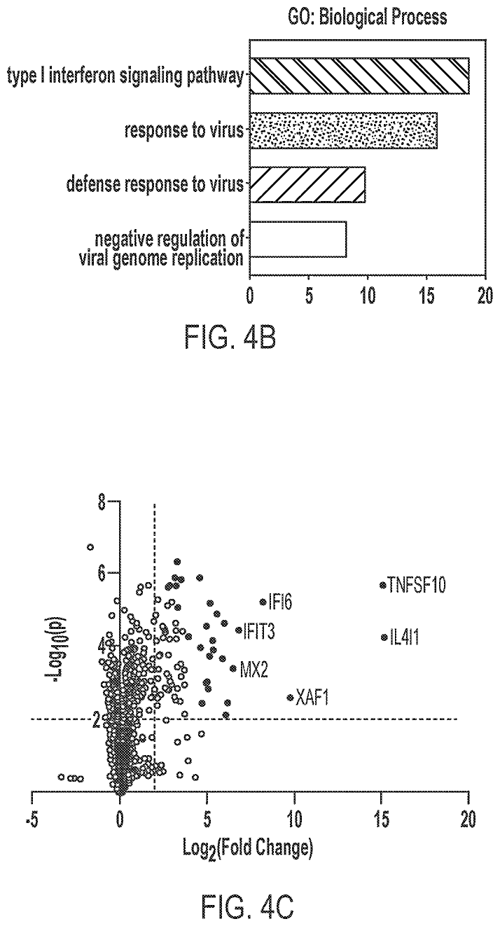

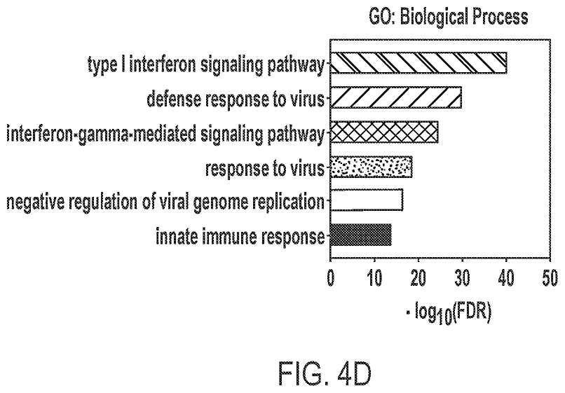

To characterize the mechanism of reduced viral infection, RNA-seq was used to characterize transcriptome changes after RNA-interference knockdown of DGCR5. Overall, 21 genes have more than a 2-fold increase with a threshold p value of 0.01 (FIG. 4A). Gene Oncology (GO) enrichment analysis reveals that the biological processes of these genes relate to type I interferon signaling pathway and the defense response to viral infections (FIG. 4B). In parallel, Tandem Mass Tag (TMT) Mass Spectrometry quantification shows upregulation of 73 proteins that have more than 4-fold increase with a threshold p value of 0.01 (FIG. 4C). GO enrichment analysis also suggests an association between knockdown of DGCR5 and upregulation of type I interferon pathways (FIG. 4D). These results indicate that DGCR5 lncRNA negatively regulates the type I interferon pathway, which explains why its knockdown suppresses influenza infection.

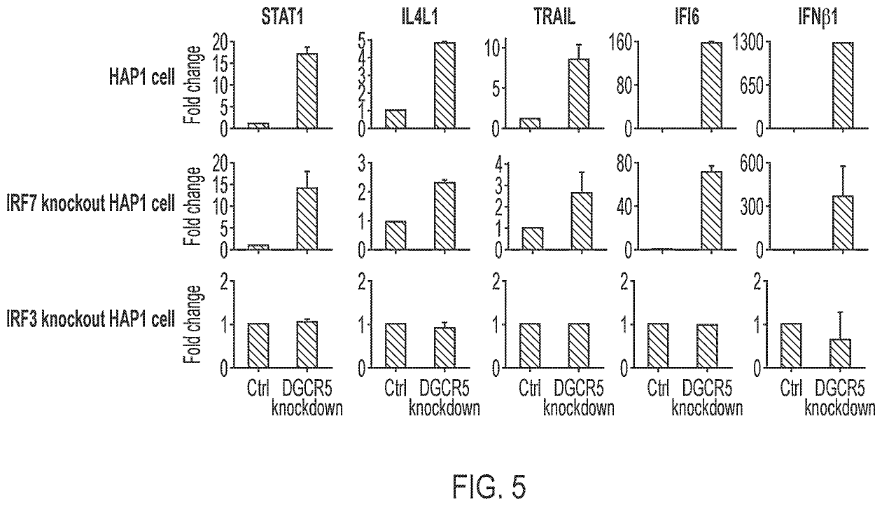

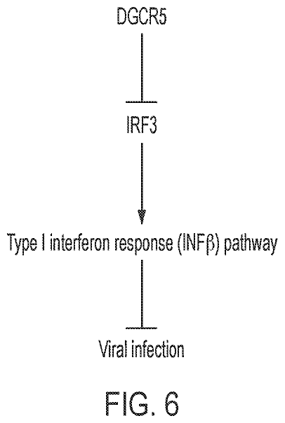

The effects of DGCR5 on type I interferon system was further explored in wild-type, interferon regulatory factor 3 (IRF3)-knockout, and IRF7-knockout HAP1 cells. IRF3 and IRF7 are transcription factors and play a vital role in interferon-I (IFN-1) production and function in viral infection⁽⁵⁾. Knockout of IRF3 rather than IRF7 abolished the effects of DGCR5 on type I interferon pathway (FIG. 5). Taken together, these results suggest that DGCR5 lncRNA negatively regulates type I interferon pathway via modulating IRF3 (FIG. 6). Given that knock down of DGCR5 can activate type I interferon pathway, DGCR5 lncRNA may be used as target for intervention in other IFN-1-associated diseases, such as infection of a broad range of viral, bacterial, fungal, and parasitic pathogens, as well as cancers autoimmune diseases, in addition to its value for influenza virus infection.

### REFERENCES

1. Zhu S, Li W, Liu J, Chen C H, Liao Q, Xu P, Xu H, Xiao T, Cao Z, Peng J, Yuan P, Brown M, Liu X S, Wei W. Genome-scale deletion screening of human long non-coding RNAs using a paired-guide RNA CRISPR-Cas9 library. Nat Biotechnol 2016; 34: 1279-1286.
2. Li W, Xu H, Xiao T, Cong L, Love M I, Zhang F, Irizarry R A, Liu J S, Brown M, Liu X S.

MAGeCK enables robust identification of essential genes from genome-scale CRISPR/Cas9 knockout screens. Genome Biol 2014; 15: 554.
3. Benam K H, Villenave R, Lucchesi C, Varone A, Hubeau C, Lee H H, Alves S E, Salmon M, Ferrante T C, Weaver J C, Bahinski A, Hamilton G A, Ingber D E. Small airway-on-a-chip enables analysis of human lung inflammation and drug responses in vitro. Nat Methods 2016; 13: 151-157.
4. Longlong Si, Rachelle Prantil-Baun, Kambez H Benam, Haiqing Bai, Melissa Rodas, Morgan Burt, Donald E. Ingber. Discovery of influenza drug resistance mutations and host therapeutic targets using a human airway chip. bioRxiv 2019; doi: doi.org/10.1101/685552.
5. Liu S, Cai X, Wu J, Cong Q, Chen X, Li T, Du F, Ren J, Wu Y T, Grishin N V, Chen Z J. Phosphorylation of innate immune adaptor proteins MAVS, STING, and TRIF induces IRF3 activation. Science 2015; 347: aaa2630.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

What is claimed is:

1. A method of inhibiting respiratory virus pathogenesis in a subject, comprising administering to a subject in need thereof an engineered nucleic acid encoding or comprising an inhibitory oligonucleotide that targets a long non-coding RNA (lncRNA), wherein the subject is infected with or at risk of infection with a respiratory virus, and wherein the lncRNA is selected from the group consisting of: DGCR5, AC015987.1, LINC01146, LRRC37A11P, LINC00176, PCAT7, CECR7, MIR503HG, RFPL1S, CYP4A22-AS1, CTC-498J12.1, RP11-360F5.1, LINC00885, LINC00086, GS1-124K5.11, CTD-2127H9.1, RP11-475N22.4, and AC108488.4.

2. A method of inhibiting respiratory virus pathogenesis in a subject, comprising administering to a subject in need thereof an engineered nucleic acid encoding or comprising an inhibitory oligonucleotide that targets a long non-coding RNA (lncRNA), wherein the subject is infected with or at risk of infection with a respiratory virus, and wherein the lncRNA is selected from the group consisting of: DGCR5, AC015987.1, LINC01146, AR, LRRC37A11P, RPL36, AAVS1, LINC00176, FOXA1, PCAT7, CECR7, RSL24D1, MIR503HG, RFPL1S, CYP4A22-AS1, RP5-107303.2, TPT1-AS1, RP11-548L20.1, LINC01060, RP1-122P22.2, AC093375.1, LINC00844, CCDC183-AS1, RP11-734K21.5, AC104135.2, CTC-527H23.3, H19, ANKRD18CP, RP11-70F11.8, RP11-167H9.6, RP6-65G23.3, RAP2C-AS1, RP11-128M1.1, RP11-76N22.2, RPL21, LINC00639, LINC00657, CTD-2541M15.1, LINC01087, MAPKAPK5-AS1, RP11-195M16.1, AC005329.7, CSAG4, RP11-760H22.2, RP1-179N16.6, RP11-333113.1, RP11-435O5.2, AC084809.2, CTD-2566J3.1, AC009478.1, CTB-181F24.1, RP11-308D16.4, RP11-314C16.1, AC020571.3, RP11-725D20.1, RP11-367G18.1, LINC01132, HOXB13, RP11-462P6.1, RP5-1142A6.9, FTX, LINC00471, RP11-498P14.5, RP11-318M2.2, CTD-2587M2.1, RP11-304F15.7, DLGAP1-AS2, RP11-299G20.2, RP11-789C1.1, RPL14, RP11-151A6.4, RP11-627G23.1, CTD-2016O11.1, ENTPD1-AS1, AE000661.37, RP11-134G8.8, SNHG5, EZH2, RPL37A, CTD-3051D23.4, LINC00925, RP11-732M18.3, JRK, RP11-802E16.3, LINC00984, EGOT, RPL39, RP11-473M20.14, TGGENE, RP11-15I11.2, RP11-677M14.3, RP11-170M17.1, RP11-65J3.1, RP11-97O12.7, SNAI3-AS1, AC095067.1, LINC01133, RP11-540A21.2, RP1-261D10.2, RP11-268G12.1, RP11-90K6.1, RP11-373N22.3, RP11-394O4.3, LINC00205, RP11-399D6.2, RP11-400K9.4, RP11-96D1.7, KB-1460A1.1, LINC00277, and RP11-269F19.2.

3. The method of any one of the preceding claims, wherein the administering upregulates a type I interferon pathway in the subject.

4. The method of any one of the preceding claims, wherein the administering inhibits pathogenesis in the subject, optionally by reducing pathogen titer.

5. The method of any one of the preceding claims, wherein the lncRNA is involved in pathogenesis of a virus.

6. The method of any one of the preceding claims, wherein the lncRNA is involved in viral propagation.

7. The method of any one of the preceding claims, wherein the virus is a respiratory virus, optionally wherein the respiratory virus is selected from the group consisting of an influenza virus (e.g., A/WSN/33 (H1N1), influenza A/Hong Kong/8/68 (H3N2), or influenza A/Avian Influenza (H5N1)), a coronavirus (e.g., betacoronavirus, e.g., SARS-CoV-2), a rhinovirus, an enterovirus, a parainfluenza virus, a metapneumovirus, a respiratory syncytial virus, an adenovirus, and a bocavirus.

8. The method of any one of the preceding claims, wherein the lncRNA is utilized by a pathogen to enhance propagation of the pathogen.

9. The method of any one of the preceding claims, wherein the lncRNA is DiGeorge Syndrome Critical Region Gene 5 (DGCR5).

10. The method of any one of the preceding claims, wherein the engineered nucleic acid comprises DNA and/or RNA.

11. The method of any one of the preceding claims, wherein the engineered nucleic acid is single stranded, double stranded, or partially double-stranded.

12. The method of any one of the preceding claims, wherein the inhibitory oligonucleotide inhibits expression and/or function of the lncRNA.

13. The method of any one of the preceding claims, wherein the inhibitory oligonucleotide binds to the lncRNA or binds to DNA encoding the lncRNA.

14. The method of any one of the preceding claims, wherein the inhibitory oligonucleotide is a clustered regularly interspaced short palindromic repeats (CRISPR) guide RNA (gRNA), optionally a Cas9 gRNA or a Cas13 gRNA.

15. The method of any one of the preceding claims, wherein the gRNA is selected from the gRNAs of Table 1 or comprises a nucleotide sequence as set forth in any one of SEQ ID NOs: 1-244.

16. The method of any one of the preceding claims, wherein the inhibitory oligonucleotide is an antisense oligonucleotide (ASO).

17. The method of any one of the preceding claims, wherein the inhibitory oligonucleotide is an RNA interference molecule.

18. The method of claim 17, wherein the RNA interference molecule is selected from the group consisting of a small interfering RNA (siRNA), a microRNA (miRNA), and a short hairpin RNA (shRNA).

19. An engineered nucleic acid encoding or comprising an inhibitory oligonucleotide that targets a long non-coding RNA (lncRNA) of Table 2, optionally for use in inhibiting respiratory virus pathogenesis.

20. The engineered nucleic acid of claim 19, wherein the lncRNA is involved in pathogenesis of a virus.

21. The engineered nucleic acid of claim 20, wherein the lncRNA is involved in viral propagation.

22. The engineered nucleic acid of claim 21, wherein the virus is a respiratory virus, optionally wherein the respiratory virus is selected from the group consisting of an influenza virus (e.g., A/WSN/33 (H1N1), influenza A/Hong Kong/8/68 (H3N2), or influenza A/Avian Influenza (H5N1)), a coronavirus (e.g., betacoronavirus, e.g., SARS-CoV-2), a rhinovirus, an enterovirus, a parainfluenza virus, a metapneumovirus, a respiratory syncytial virus, an adenovirus, and a bocavirus.

23. The engineered nucleic acid of any one of claims 19-22, wherein the lncRNA is utilized by a pathogen to enhance propagation of the pathogen.

24. The engineered nucleic acid of any one of claims 19-23, wherein the lncRNA is selected from the group consisting of: DGCR5, AC015987.1, LINC01146, LRRC37A11P, LINC00176, PCAT7, CECR7, MIR503HG, RFPL1S, CYP4A22-AS1, CTC-498J12.1, RP11-360F5.1, LINC00885, LINC00086, GS1-124K5.11, CTD-2127H9.1, RP11-475N22.4, AC108488.4, and TMEM44-AS1.

25. The engineered nucleic acid of any one of claims 19-24, wherein the lncRNA is selected from the group consisting of: DGCR5, AC015987.1, LINC01146, AR, LRRC37A11P, RPL36, AAVS1, LINC00176, FOXA1, PCAT7, CECR7, RSL24D1, MIR503HG, RFPL1S, CYP4A22-AS1, RP5-107303.2, TPT1-AS1, RP11-548L20.1, LINC01060, RP1-122P22.2, AC093375.1, LINC00844, CCDC183-AS1, RP11-734K21.5, AC104135.2, CTC-527H23.3, H19, ANKRD18CP, RP11-70F11.8, RP11-167H9.6, RP6-65G23.3, RAP2C-AS1, RP11-128M1.1, RP11-76N22.2, RPL21, LINC00639, LINC00657, CTD-2541M15.1, LINC01087, MAPKAPK5-AS1, RP11-195M16.1, AC005329.7, CSAG4, RP11-760H22.2, RP1-179N16.6, RP11-333113.1, RP11-435O5.2, AC084809.2, CTD-2566J3.1, AC009478.1, CTB-181F24.1, RP11-308D16.4, RP11-314C16.1, AC020571.3, RP11-725D20.1, RP11-367G18.1, LINC01132, HOXB13, RP11-462P6.1, RP5-1142A6.9, FTX, LINC00471, RP11-498P14.5, RP11-318M2.2, CTD-2587M2.1, RP11-304F15.7, DLGAP1-AS2, RP1-299G20.2, RP11-789C1.1, RPL14, RP11-151A6.4, RP11-627G23.1, CTD-2016O11.1, ENTPD1-AS1, AE000661.37, RP11-134G8.8, SNHG5, EZH2, RPL37A, CTD-3051D23.4, LINC00925, RP11-732M18.3, JRK, RP11-802E16.3, LINC00984, EGOT, RPL39, RP11-473M20.14, TGGENE, RP11-15I11.2, RP11-677M14.3, RP11-170M17.1, RP11-65J3.1, RP11-97O12.7, SNAI3-AS1, AC095067.1, LINC01133, RP11-540A21.2, RP1-261D10.2, RP11-268G12.1, RP11-90K6.1, RP11-373N22.3, RP11-394O4.3, LINC00205, RP11-399D6.2, RP11-400K9.4, RP11-96D1.7, KB-1460A1.1, LINC00277, and RP11-269F19.2.

26. The engineered nucleic acid of any one of claims 19-25, wherein the lncRNA is DiGeorge Syndrome Critical Region Gene 5 (DGCR5).

27. The engineered nucleic acid of any one of claims 19-26, wherein the inhibitory oligonucleotide is a clustered regularly interspaced short palindromic repeats (CRISPR) guide RNA (gRNA), optionally a Cas9 gRNA or a Cas13 gRNA.

28. The engineered nucleic acid of claim 27, wherein the gRNA is selected from the gRNAs of Table 1 or comprises a nucleotide sequence as set forth in any one of SEQ ID NOs: 1-244.

29. A vector comprising the engineered nucleic acid of any one of the preceding claims, optionally wherein the vector is selected from the group consisting of a plasmid, a phagemid, a cosmid, and a viral vector.

30. A nanoparticle comprising the engineered nucleic acid of any one of the preceding claims, optionally wherein the nanoparticle is a lipid nanoparticle.

31. A pharmaceutical composition comprising the engineered nucleic acid, vector, or nanoparticle of any one of the preceding claims and a pharmaceutically-acceptable excipient.

32. A method comprising administering to a subject the engineered nucleic acid, vector, nanoparticle, or pharmaceutical composition of any one of the preceding claims, optionally wherein the subject is infected with or at risk of infection with a pathogen.
